(19) United States
(12) Patent Application Publication
(10) Pub. No.: US 2026/0168025 A1
(43) Pub. Date: Jun. 18, 2026

(54) BIOMARKER SET IDENTIFICATION FOR LYME DISEASE

(71) Applicants: ICAHN SCHOOL OF MEDICINE AT MOUNT SINAI, New York, NY (US); THE JOHNS HOPKINS UNIVERSITY, Baltimore, MD (US)

(72) Inventors: Avi Maayan, New York, NY (US); Daniel Clark, New York, NY (US); Alison W. Rebman, Baltimore, MD (US); John N. Aucott, Baltimore, MD (US); Mark J. Soloski, Baltimore, MD (US)

(21) Appl. No.: 18/858,000

(22) PCT Filed: Apr. 28, 2023
(86) PCT No.: PCT/US2023/020424

§ 371 Date: Oct. 18, 2024

Related U.S. Application Data
(60) Provisional application No. 63/336,762, filed on Apr. 29, 2022.

(51) Int. Cl. C12Q 1/6883; C12Q 1/689; G16B 25/10; G16B 40/20

(52) U.S. Cl. CPC C12Q 1/6883; C12Q 1/689; G16B 25/10; G16B 40/20; C12Q 2600/158

(57) ABSTRACT

The presently claimed and described technology provides methods of detecting Lyme disease or post-treatment Lyme disease by identifying changes in expression levels of at least one mRNA present in a biological sample, comparing the at least one mRNA's expression level with the levels of the same mRNA of a reference sample or control sample and diagnosing Lyme disease or post-treatment Lyme disease based on changes in the expression levels.

### RELATED APPLICATIONS

The present patent application claims the priority benefit of U.S. Provisional Patent Application Ser. No. 63/336,762, filed Apr. 29, 2022, the content of which is hereby incorporated by reference in its entirety into this disclosure.

### STATEMENT REGARDING FEDERALLY SPONSORED RESEARCH OR DEVELOPMENT

This was made with government support under P30AR070254 by the National Institute of Arthritis and Musculoskeletal and Skin Diseases. The government has certain rights.

### BACKGROUND

Lyme disease (LD) is a tick-borne disease whose post-treatment sequelae are not well-understood. Approximately 30,000 diagnosed cases of Lyme disease (LD) are reported to the CDC each year. However, the actual estimated burden is ˜476,000 cases, carrying a yearly healthcare cost of ˜$1 billion in the US. Testing and diagnosis of the earliest stages of LD have proven to be difficult or unreliable. The universally accepted diagnostic test for LD is a positive enzyme-linked immunosorbent assay (ELISA) followed by a positive Western blot for IgM and IgG B referred to as the two-tier test (TTT). In addition, there is a recently introduced, modified two-tier test (MTTT) and a test for antibodies reactive to the VlsE1 antigen. The TTT test has a sensitivity of 17%-43% during the early stage of infection. In the absence of a laboratory diagnostic tool, the diagnosis of early LD is reliant on clinical demonstration of the erythema migrans (EM) skin lesion that occasionally does not present or is not observed. This can lead patients to progress to early disseminated or late-stage disease, which can have more difficult-to-treat symptoms, before the disease is diagnosed and treated with antibiotics. Antibiotic treatment includes a dosing regimen of doxycycline, amoxicillin, ceftriaxone, or cefotaxime, dependent on the patient age and displayed symptoms. Even when the disease is clearly diagnosed and properly treated, about 10%-20% of patients do not respond completely and develop prolonged symptoms, a condition termed post-treatment Lyme disease (PTLD). According to the proposed case definition put forth in the 2006 guidelines of the Infectious Diseases Society of America (IDSA), PTLD is characterized by a previously documented case of LD infection, completion of appropriate antibiotics, and symptoms six months after completion of antibiotic treatment of fatigue, bodily pain, and/or cognitive difficulties which impact day-to-day life. In 2020, the IDSA updated these guidelines; however, this proposed research case definition for PTLD was removed. PTLD has been controversial in the medical community due to its non-characteristic symptoms and the current inability to identify the causes of the persistent symptoms and their subsequent resolution. While prior studies have shown altered biology in patients with PTLD, there are no biomarkers to diagnose the condition.

Several studies have examined the gene expression profile of cells and tissues from patients with acute LD. In the studies examining peripheral blood mononuclear cells (PBMCs), all three studies identified strong differential gene expression (DRG) signatures during acute disease that were differentiated from healthy controls, and these signatures were dominated by the expression of numerous immune-related genes. In two of these three studies, when gene expression was examined at later time points after antibiotic treatment, the DRG signature could be distinguished from healthy controls up to one-year post-infection, even though symptoms had largely resolved in many cases. The underlying mechanisms that drive this sustained gene expression are not clear. However, in a third study, it was observed that by six months after antibiotic treatment, the gene expression signatures of Lyme and heathy control cases were indistinguishable. Presently, the reason for this discrepancy is not clear but may relate to the size and/or composition of the cohorts. Importantly, all three studies were able to identify LD gene signatures that may be of value in the diagnosis and staging of human LD. The study examining gene expression within whole EM tissue identified several immune related genes, including cytokines, chemokines, TLRs, antimicrobial peptides, interferon inducible genes, and genes associated with monocytoid cell activation. More recently, single cell transcriptome analysis of cells recovered from EM lesions clearly identified antigen driven clonal expansion of novel B cell subsets, as well as the presence of activated T cells and myeloid subsets. Collectively, these studies demonstrate that infection with B. burgdorferi is accompanied by activation of cellular elements of the innate and adaptive immune response and that can be identified locally in tissue and in the blood.

To further the understanding of the molecular mechanisms that may contribute to PTLD symptoms and to identify reliable biomarkers for the diagnosis of PTLD, transcriptional profiles of PBMCs isolated from 152 patients with PTLD were examined and these profiles compared to patients with acute LD and uninfected healthy control participants. Visualization of these cohorts was performed by examining the projection of the RNA-seq profiles into lower dimensions. In addition, differential gene expression analysis followed by enrichment analysis was employed to identify upstream regulatory mechanisms and disease phenotypes distinctly associated with LD and PTLD. Next, the differentially expressed genes were further analyzed to identify genes that may serve as an mRNA biomarker to confirm LD at the early stages of the disease, as well as assist in distinguishing between completely convalescent patients and those with PTLD.

### BRIEF SUMMARY

One aspect of the disclosure is a method of detecting Lyme disease or post-treatment Lyme disease in a subject in need thereof, the method including identifying changes in expression levels of at least one mRNA present in a biological sample, wherein the biological sample was obtained from a subject having, suspected of having, or at risk of having Lyme disease or post-treatment Lyme disease; comparing the at least one mRNA's expression level with the levels of the same mRNA of a reference sample or control sample; and wherein changes in the expression levels of the subject's mRNA from those of the reference sample or control sample correlates with a diagnosis of Lyme disease or post-treatment Lyme disease.

In an aspect, the method further includes calculating a score for the at least one mRNA and assigning a classification to the at least one mRNA based on the score. In another aspect, the method further including predicting a likelihood of the subject having Lyme disease or post-treatment Lyme disease based on the score.

One aspect of the disclosure is a computer-implemented method for detecting Lyme disease or post-treatment Lyme disease in a subject in need thereof, the method including obtaining mRNA expression data for a biological sample, wherein the biological sample was obtained from a subject having, suspected of having, or at risk of having Lyme disease or post-treatment Lyme disease; identifying, with one or more computing devices, changes in expression levels of at least one mRNA present in a biological sample; comparing, with one or more computing devices, the at least one mRNA's expression level with the levels of the same mRNA of a reference sample or control sample; and calculating, with one or more computing devices, a likelihood of the subject having Lyme disease or post-treatment Lyme disease based on the comparison of the expression levels.

In an aspect, the method further includes using one or more computing devices to assign a threshold value to the at least one mRNA. In another aspect, the identifying, comparing, and/or calculating step may be modified based on at least one machine learning algorithm.

In an aspect, the at least one mRNA is an upregulated mRNA or a downregulated mRNA. In an aspect, the biological sample is selected from the group consisting of whole blood, plasma, peripheral blood mononuclear cells, serum, lymph, cerebrospinal fluid, ascites, and tissue biopsy. In an aspect the control sample is from a healthy subject or a subject with acute Lyme disease.

In an aspect, the mRNA is selected from the group including KLHL11, UTF1, NBPF1, RBMS3, PPFIA4, NOTCH3, SLC4A10, TMEM272, CBARP, MLANA, CHDH, NAP1L2, TMEM52B, C2CD4D, OTUB2, POMK, KCNG1, CAND2, DCSTAMP, NBEAL1, SCN3A, AHNAK2, RAD50, APBA1, DNAH7, CXADR, AMH, ALDH7A1, CACNB4, CMTM1, SHANK1, TULP2, NEK5, BTNL9, and GPR135.

In an aspect, the method includes identifying changes in expression levels of at two mRNAs, alternatively at least three mRNAs, alternatively at least four mRNAs, alternatively at least five mRNAs, alternatively at least six mRNAs, alternatively at least seven mRNAs, alternatively at least eight mRNAs, alternatively at least nine mRNAs, alternatively at least ten mRNAs, alternatively at least eleven mRNAs, alternatively at least twelve mRNAs, alternatively at least thirteen mRNAs, alternatively at least fourteen mRNAs, alternatively at least fifteen mRNAs, alternatively at least sixteen mRNAs, alternatively at least seventeen mRNAs, alternatively at least eighteen mRNAs, alternatively at least nineteen mRNAs, alternatively at least twenty mRNAs, alternatively at least twenty-one mRNAs, alternatively at least twenty-two mRNAs, alternatively at least twenty-three mRNAs, alternatively at least twenty-four mRNAs, alternatively at least twenty-five mRNAs, alternatively at least twenty-six mRNAs, alternatively at least twenty-seven mRNAs, alternatively at least twenty-eight mRNAs, alternatively at least twenty-nine mRNAs, alternatively at least thirty mRNAs, alternatively at least thirty-one mRNAs, alternatively at least thirty-two mRNAs, alternatively at least thirty-three mRNAs, alternatively at least thirty-four mRNAs, or alternatively at least thirty-five mRNAs. In another aspect, the method includes identifying changes in expression levels of KLHL11 and UTF1.

On aspect of the disclosure is a system for detecting Lyme disease or post-treatment Lyme disease in a subject in need thereof, the system including: one or more computing devices, and a memory having instructions stored thereon, wherein the instructions, when executed by the one or more computing devices, cause the one or more computing devices to identify changes in expression levels of at least one mRNA present in a biological sample, wherein the biological sample was obtained from a subject having, suspected of having, or at risk of having Lyme disease or post-treatment Lyme disease; compare the at least one mRNA's expression level with the levels of the same mRNA of a reference sample or control sample; and calculate a likelihood of the subject having Lyme disease or post-treatment Lyme disease based on the comparison of the expression levels.

In an aspect, the system is configured to provide defined artificial intelligence (AI) sensing and autonomous response. In another aspect of the system, the at least one mRNA is an upregulated mRNA or a downregulated mRNA. In an aspect of the system, the biological sample is selected from the group consisting of whole blood, plasma, peripheral blood mononuclear cells, serum, lymph, cerebrospinal fluid, ascites, and tissue biopsy. In another aspect of the system, the control sample is from a healthy subject or a subject with acute Lyme disease.

In an aspect of the system, the mRNA is selected from the group including KLHL11, UTF1, NBPF1, RBMS3, PPFIA4, NOTCH3, SLC4A10, TMEM272, CBARP, MLANA, CHDH, NAP1L2, TMEM52B, C2CD4D, OTUB2, POMK, KCNG1, CAND2, DCSTAMP, NBEAL1, SCN3A, AHNAK2, RAD50, APBA1, DNAH7, CXADR, AMH, ALDH7A1, CACNB4, CMTM1, SHANK1, TULP2, NEK5, BTNL9, and GPR135.

In an aspect, the system includes identifying changes in expression levels of at least two mRNAs, alternatively at least three mRNAs, alternatively at least four mRNAs, alternatively at least five mRNAs, alternatively at least six mRNAs, alternatively at least seven mRNAs, alternatively at least eight mRNAs, alternatively at least nine mRNAs, alternatively at least ten mRNAs, alternatively at least eleven mRNAs, alternatively at least twelve mRNAs, alternatively at least thirteen mRNAs, alternatively at least fourteen mRNAs, alternatively at least fifteen mRNAs, alternatively at least sixteen mRNAs, alternatively at least seventeen mRNAs, alternatively at least eighteen mRNAs, alternatively at least nineteen mRNAs, alternatively at least twenty mRNAs, alternatively at least twenty-one mRNAs, alternatively at least twenty-two mRNAs, alternatively at least twenty-three mRNAs, alternatively at least twenty-four mRNAs, alternatively at least twenty-five mRNAs, alternatively at least twenty-six mRNAs, alternatively at least twenty-seven mRNAs, alternatively at least twenty-eight mRNAs, alternatively at least twenty-nine mRNAs, alternatively at least thirty mRNAs, alternatively at least thirty-one mRNAs, alternatively at least thirty-two mRNAs, alternatively at least thirty-three mRNAs, alternatively at least thirty-four mRNAs, or alternatively at least thirty-five mRNAs. In another aspect, the system includes identifying changes in expression levels of KLHL11 and UTF1.

One aspect of the disclosure includes one or more non-transitory computer-readable storage media comprising instructions, which when executed by one or more computing devices, cause the one or more computing devices to: identify changes in expression levels of at least one mRNA present in a biological sample, wherein the biological sample was obtained from a subject having, suspected of having, or at risk of having Lyme disease or post-treatment Lyme disease; compare the at least one mRNA's expression level with the levels of the same mRNA of a reference sample or control sample; and calculate a likelihood of the subject having Lyme disease or post-treatment Lyme disease based on the comparison of the expression levels.

These and other advantages, aspects, and novel features of the present disclosure, as well as details of an illustrated embodiment thereof, will be more fully understood from the following description and drawings.

### BRIEF DESCRIPTION OF THE DRAWINGS

Various aspects of the present disclosure will now be described, by way of example only, with reference to the attached Figures, wherein:

FIGS. 1A and 1B are PCA of normalized RNA-seq expression vectors labeled by cohort. A number of samples in each group are annotated in the figure legend. Acute LD refers to the cohort of patients at their initial visit; healthy refers to non-Lyme exposed healthy control participants; and PTLD refers to the cohort of patients diagnosed with post-treatment Lyme disease. FIG. 1C is a Venn diagram showing the differences in mRNA biomarker gene makeup from the genes proposed (new) and those proposed by applying the entire pipeline with PTLD patients with LD less than six months removed (old).

FIGS. 2A-2D illustrate enrichment analysis of differentially expressed genes (DEGs). FIG. 2A illustrates enrichment results from Enrichr for significantly DEGs in controls versus PTLD up (up in PTLD). FIG. 2B illustrates enrichment results from Enrichr for significantly DEGs in controls versus PTLD down (down in PTLD). FIG. 2C illustrates enrichment results from Enrichr for significantly DEGs in acute LD versus PTLD up (up in PTLD). FIG. 2D illustrates enrichment results from Enrichr for significantly DEGs in acute LD versus PTLD down (down in PTLD).

FIG. 3 is a Venn diagram showing differentially expressed genes in acute LD and PTLD compared with the KEGG term herpes simplex virus 1 infection.

FIG. 4 is a Super-Venn diagram visualization of overlapping gene sets between the cohorts and genes known to be related to inflammatory responses in other diseases. Counts on the top correspond to the number of sets that overlap, counts on the right correspond to the number of genes in the set, and counts at the bottom correspond to the number of genes in the intersecting set. Up- and downregulated genes in acute LD refer to significantly DEGs with respect to the healthy individuals at visit 1. Up- and downregulated genes in PTLD refer to significantly DEGs with respect to the healthy individuals. Viral, bacterial, and spirochete genes derived from Enrichr gene sets and filtered by genes that are also differentially expressed in LD or PTLD.

FIGS. 5A-5C illustrate the performance of the classifier using 35 mRNA biomarker genes chosen with the training set. Performance is based on training four independent models and predicting the held-out samples not used during training or feature selection. The test set is randomly under-sampled such that classes are balanced. p values are based on a permutation test across different potential train-test splits. FIG. 5A shows confusion matrices for a two-gene classifier. FIG. 5B shows confusion matrices for the 35-gene biomarker set. FIG. 5C is ROC and Precision Recall curves for the 35-gene-set biomarker.

FIG. 6 illustrates enrichment results from Enrichr for the 35 biomarker genes.

FIG. 7A is a heat map illustrating classifier performance with single genes. Performance of singular mRNA biomarkers as the feature for the models. Performance is based on training four independent models with the training set and predicting the test samples not used during training. The test set is randomly under-sampled such that classes are balanced. AUROCs for each gene are reported as a heatmap.

FIG. 7B illustrates classifier performance using only the two mRNA biomarker genes KLHL11 and UTF1 as features for the models. Performance is based on training four independent models with the training set and predicting the test samples not used during training. The test set is randomly under-sampled such that classes are balanced. P-values are based on a permutation test across different potential train-test splits.

FIG. 8 shows an example aspect of a computing device.

### DETAILED DESCRIPTION

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

The invention is defined in the claims. However, below is a non-exhaustive listing of non-limiting exemplary aspects. Any one or more of the features of these aspects may be combined with any one or more features of another example, embodiment, or aspect described herein.

LD and the infection-associated sequelae of PTLD continue to emerge as an important public health issue. Existing tests to confirm diagnosis have limited accuracy, especially in supporting the diagnosis of PTLD, which is made in persistently symptomatic patients many months to years after appropriate diagnosis and treatment. Here the whole PBMC transcriptome analysis was applied to a set of individuals in a well-defined cross-sectional cohort of patients with PTLD. The PTLD signature was found to be distinct from the healthy controls and from individuals diagnosed with acute LD, which were part of a longitudinal cohort that has been reported on previously. Importantly, the PTLD and acute LD RNA-seq signatures were sufficiently distinct, enabling us to design a set of mRNA markers that will be of value in distinguishing acute LD, PTLD, and healthy controls. Although the patients diagnosed with PTLD have a similar immune activation to the patients with acute LD, there is a component of this immune response that is diminished or altered. The reduction in immune activation is expected, but the observation that these patients are markedly different from healthy controls is illuminating. The separation of participants with PTLD from the healthy controls and acute LD clusters may be explained by batch effects. However, removing the batch effects is difficult because the group labels correspond to the batch labels. The observation that the differentially expressed genes point to bacterial infection and inflammatory response suggests that the differences observed are not just due to batch effects. Some of the differentially expressed gene in PTLD point to common symptoms observed in these patients. For example, neuropsychiatric symptoms that have been reported in patients with PTLD and in patients with Lyme encephalopathy are consistent with the enrichment analysis, which suggests the potential genetic underpinning of such a phenotype. Expression data from RNA-seq applied to PBMCs collected from acute LD, PTLD, and healthy controls yield distinct separation of patients along the first two principal component axes of variance. This suggests that mRNA biomarkers may be feasibly identified to diagnose acute LD and PTLD. Gene set overlap analysis was used to identify consensus and divergent differentially expressed genes in acute LD and PTLD and compared to gene sets from other viral and bacterial infections. The resulting genes that are specific for LD were further reduced and classifiers were constructed to assess the feasibility of developing a diagnostic. Overall, the gene classifiers identified can categorize patients with acute LD and PTLD well using as few as two mRNA biomarkers.

One aspect of the disclosure is a method of detecting Lyme disease or post-treatment Lyme disease in a subject in need thereof. This method includes identifying changes in expression levels of at least one mRNA present in a biological sample, wherein the biological sample was obtained from a subject having, suspected of having, or at risk of having Lyme disease or post-treatment Lyme disease. In non-limiting examples, the is selected from the group including whole blood, peripheral blood mononuclear cells, plasma, serum, lymph, cerebrospinal fluid, ascites, and tissue biopsy.

The method further includes comparing the at least one mRNA's expression level with the levels of the same mRNA of a reference sample or control sample wherein changes in the expression levels of the subject's mRNA from those of the reference sample or control sample correlates with a diagnosis of Lyme disease or post-treatment Lyme disease.

The mRNA from the subject may be an upregulated gene, meaning that the mRNA is expressed in greater amounts in the biological sample as compared to the level of the same mRNA in a reference sample or a control sample. The mRNA may be a down regulated gene, meaning that the mRNA is expressed in lower amounts in the biological sample as compared to the level of the same mRNA in a reference sample or a control sample.

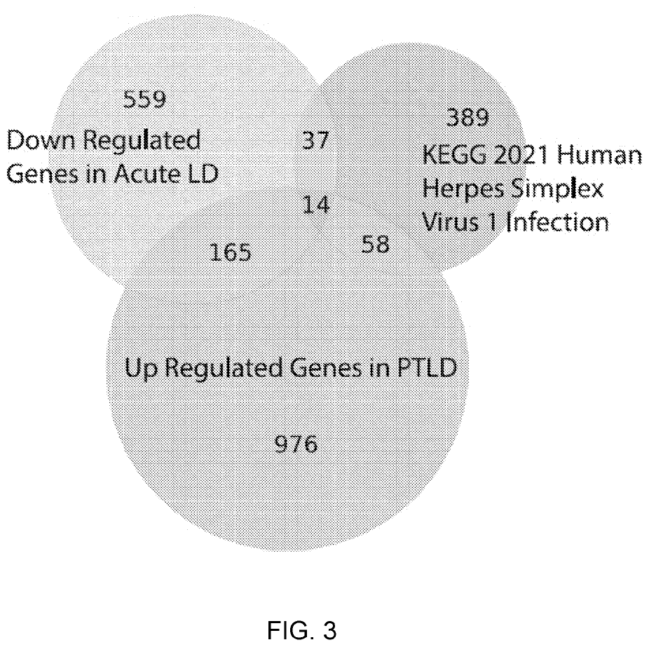

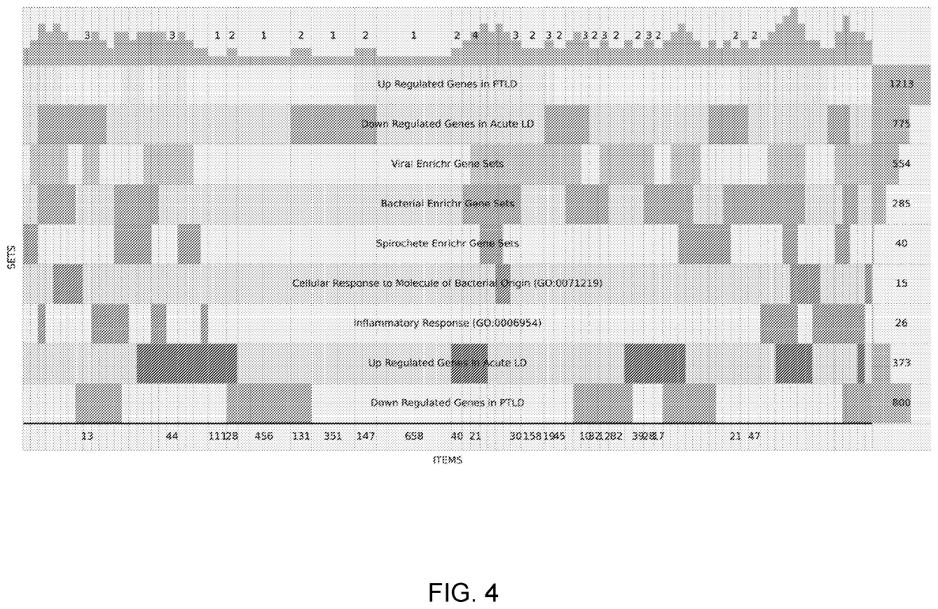

It will be appreciated by one of ordinary skill that several mRNA expression levels can be evaluated at once. In various embodiments, the method includes identifying changes in expression levels of at two mRNAs, alternatively at least three mRNAs, alternatively at least four mRNAs, alternatively at least five mRNAs, alternatively at least six mRNAs, alternatively at least seven mRNAs, alternatively at least eight mRNAs, alternatively at least nine mRNAs, alternatively at least ten mRNAs, alternatively at least eleven mRNAs, alternatively at least twelve mRNAs, alternatively at least thirteen mRNAs, alternatively at least fourteen mRNAs, alternatively at least fifteen mRNAs, alternatively at least sixteen mRNAs, alternatively at least seventeen mRNAs, alternatively at least eighteen mRNAs, alternatively at least nineteen mRNAs, alternatively at least twenty mRNAs, alternatively at least twenty-one mRNAs, alternatively at least twenty-two mRNAs, alternatively at least twenty-three mRNAs, alternatively at least twenty-four mRNAs, alternatively at least twenty-five mRNAs, alternatively at least twenty-six mRNAs, alternatively at least twenty-seven mRNAs, alternatively at least twenty-eight mRNAs, alternatively at least twenty-nine mRNAs, alternatively at least thirty mRNAs, alternatively at least thirty-one mRNAs, alternatively at least thirty-two mRNAs, alternatively at least thirty-three mRNAs, alternatively at least thirty-four mRNAs, or alternatively at least thirty-five mRNAs. In an aspect, these multiple mRNAs may all be upregulated genes, all be downregulated genes, or a combination thereof. For example, FIGS. 3 and 4 show various upregulated and downregulated genes that may be used in the disclosed methods.

A reference sample refers to a reference standard wherein the mRNA expression level is constant. In some embodiments, the mRNA expression level may be the same or substantially similar to mRNA expression levels found in healthy humans. The reference sample may be obtained from a database, such as, for example, a RNA-seq database, a gene expression database, or a genotype-tissue expression database. The control sample may be generated from a control sample is from a healthy subject or a subject with acute Lyme disease. The control sample may be obtained from a database. Additionally, the expression levels in both the control sample and the biological sample from the subject may be identified using any known technique in the art for measuring mRNA levels, including, but not limited to, northern blotting, microarray analysis, and reverse transcription polymerase chain reaction (RT-PCT).

In an aspect, the method further includes calculating a score for the at least one mRNA and assigning a classification to the at least one mRNA based on the score. In an embodiment, the classification may be high, medium, or low. The method may further include predicting a likelihood of the subject having Lyme disease or post-treatment Lyme disease based on the score and/or classification.

One aspect of the disclosure is a computer-implemented method for detecting Lyme disease or post-treatment Lyme disease in a subject in need thereof, the method includes obtaining mRNA expression data for a biological sample, wherein the biological sample was obtained from a subject having, suspected of having, or at risk of having Lyme disease or post-treatment Lyme disease. Using the one or more computing devices, the method includes identifying, with one or more computing devices, changes in expression levels of at least one mRNA present in a biological sample; comparing the at least one mRNA's expression level with the levels of the same mRNA of a reference sample or control sample; and calculating a likelihood of the subject having Lyme disease or post-treatment Lyme disease based on the comparison of the expression levels.

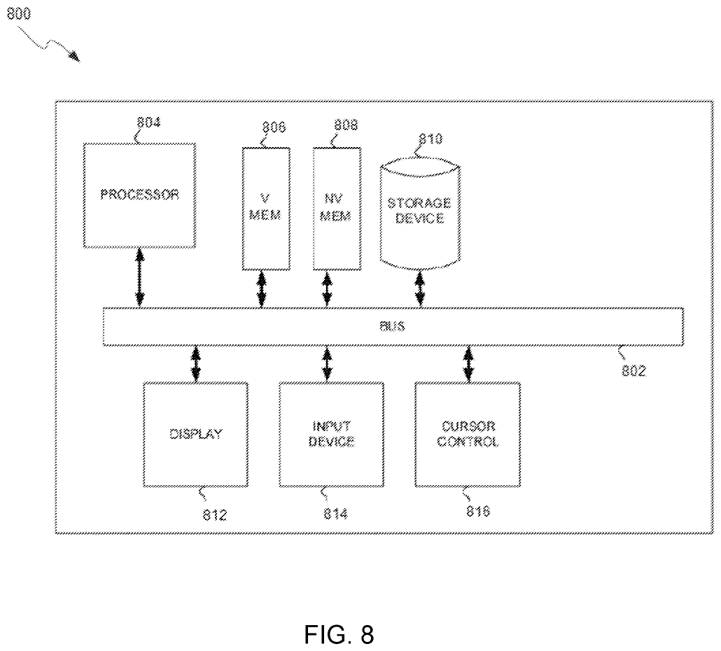

The method may include using one or more computing devices to assign a threshold value to the at least one mRNA. An exemplary computing device is illustrated in FIG. 8. In some examples, the threshold value(s) and/or range of thresholds may be configurable by a user and may vary based on the mRNA identified. The threshold value(s) and/or range of thresholds may also be modified based on machine learning algorithms. For example, in some embodiments, the identifying, comparing, and/or calculating step may be modified based on at least one machine learning algorithm.

Various mRNA may be used in the method. In a non-limiting example, an mRNA based diagnostic biomarker panel may include one or more of KLHL11, UTF1, NBPF1, RBMS3, PPFIA4, NOTCH3, SLC4A10, TMEM272, CBARP, MLANA, CHDH, NAP1L2, TMEM52B, C2CD4D, OTUB2, POMK, KCNG1, CAND2, DCSTAMP, NBEAL1, SCN3A, AHNAK2, RAD50, APBA1, DNAH7, CXADR, AMH, ALDH7A1, CACNB4, CMTM1, SHANK1, TULP2, NEK5, BTNL9, and GPR135. In one exemplary embodiment, the method includes identifying changes in expression levels of KLHL11 and UTF1.

The disclosure also provides a system for detecting Lyme disease or post-treatment Lyme disease in a subject in need thereof, the system including one or more computing devices, and a memory having instructions stored thereon, wherein the instructions, when executed by the one or more computing devices, cause the one or more computing devices to identify changes in expression levels of at least one mRNA present in a biological sample, wherein the biological sample was obtained from a subject having, suspected of having, or at risk of having Lyme disease or post-treatment Lyme disease, compare the at least one mRNA's expression level with the levels of the same mRNA of a reference sample or control sample, and calculate a likelihood of the subject having Lyme disease or post-treatment Lyme disease based on the comparison of the expression levels.

In some aspects, the system may be configured to provide defined artificial intelligence (AI) sensing and autonomous response. This may be particularly possible and/or optimized in conjunction with the extending of network edges and/or accessibility to cloud resources. Such AI based solution may include and/or entails the use of AI sensing, AI autonomy, and AI cloud services. AI sensing or machine learning models may be used to estimate several characteristics of the mRNA expression and regulation. AI software may also use AI deep learning. The software may integrate data from third parties and other sources as needed. With respect to AI cloud services, users may use AI cloud services or solutions (e.g., cloud-based software applications) to run their data. For example, an AI or learning algorithm may be used to analyze mRNA expression data and to build a prediction model for Lyme disease or post-treatment Lyme disease of appendicitis based on relevant biomarkers. In an embodiment, a trained model is developed using mRNA expression data. After developing the trained model, the model may be used to predict a subject's diagnosis using class probability. Class probabilities may incorporate a decision boundary, wherein suspected positive/suspected negative samples are identified in full or in part based on the decision boundary.

The disclosure also provides for one or more non-transitory computer-readable storage media comprising instructions, which when executed by one or more computing devices, cause the one or more computing devices to: identify changes in expression levels of at least one mRNA present in a biological sample, wherein the biological sample was obtained from a subject having, suspected of having, or at risk of having Lyme disease or post-treatment Lyme disease; compare the at least one mRNA's expression level with the levels of the same mRNA of a reference sample or control sample; and calculate a likelihood of the subject having Lyme disease or post-treatment Lyme disease based on the comparison of the expression levels.

The results suggest the possibility of utilizing an mRNA based diagnostic biomarker panel, in combination with precise clinical evaluations, to identify and/or categorize patients in whom Lyme disease is suspected. This approach could be adapted to utilize whole blood, a readily accessible tissue and would not rely on the detection of anti-Borrelia antibodies or bacterial DNA, approaches that have been shown to lack sensitivity. In addition, previous studies have shown that levels of chemokines (CCL19), serum metabolites, and a fecal microbiome signature have been associated with the development of PTLD19-21. Therefore, it is possible that an approach incorporating mRNA combined with other molecular signatures may lead to an accurate diagnostic with high sensitivity and specificity in diagnosing the multiple stages of human LD, including PTLD. This, however, will require a broader study design using samples from multiple PTLD cohorts as well as the study of cohorts from other disorders such as post-acute COVID-19 and chronic fatigue syndrome.

The finding that PBMCs from patients with PTLD express an mRNA signature also indicates that PTLD has a specific underlying biology. Understanding this complex biology will be of great value in the development of novel treatment strategies. The gene expression data on this large group of patients with PTLD expands on previous work, all of which lead to the identification of gene classifiers for acute LD8-10. In addition, the studies on acute LD identified a mRNA signature that was consistent with a strong immune response. In this study which focuses on PTLD, analysis using a SuperVenn showed that both up and down regulated genes overlapped with host inflammatory response genes, and genes linked to viral and bacterial infections. Comparing the genes that distinguish PTLD from acute Lyme identified several immune features. The complement pathway is identified using KEGG22 and BioCarta pathways analysis, as well as GO Biological Processes23 enrichment analysis. In addition, enrichment analysis using the Azimuth Cell Types library identified gene signatures from immune cell types including plasmablasts and proliferating CD4+ and CD8+ T cells. This would imply, as mentioned above, that several immune pathways are a part of the underlying biology of PTLD.

In addition, the analysis also identified non-immune features. For example, when comparing upregulated genes in PTLD versus healthy controls, WikiPathways24 enrichment analysis identified a gene set related to primary cilium development and ciliopathies and GO Cellular Components revealed cilium (GO: 0005929) and motile cilium (GO: 0031514). These are features that are associated with non-immune, non-bone marrow derived cell types such as epithelial cells. Of note, cell type enrichment analysis using the Descartes Cell Types and Tissue 2021 library identified a signature associated with ciliated epithelial cells of the lung and the Human Gene Atlas library identified enriched terms aligned with non-immune cell types. This signature could be from a non-bone marrow derived cell that bears primary cilia, or it can be from an immune cell that expresses cilia-like projection. Regarding this latter possibility, recent data has shown that effector T cells can form cilia-like projections, most notably in the process of immune synapse formation when effector T cells are interacting with targets cells. These structures have been referred to as “frustrated cilia” and the genes responsible overlap considerably with genes known to be involved in formation of primary cilia in epithelium and other non-immune cell types25. These genes include TTC26, TTC23, IFT 74, IFT81, IFT85, ARL13B, CEP83, CEP162, CEP76, and CEP44. CEP83 encodes a protein involved in centrosome docking on the plasma membrane and is critical for primary cilia and immune synapse formation. ARL13B encodes a guanine exchange factor, which regulates membrane composition and the recruitment of signaling molecules in both the cilium and immune synapses. TTC genes encode tetratricopeptide repeat-containing proteins that can interact with IFT proteins and are critical for cilia formation and function28. Intraflagellar transport (IFT) proteins participate in the active sorting and transport of cytosolic and membrane proteins destined for the cilium and this can include signaling molecules29. All these genes are upregulated in PTLD patients relative to healthy controls suggesting the present of an immune cell type in circulation that is in the process of assembling cilia-like structures. This is consistent with the discussion above suggesting that immune activation is a component of the underlying biology of PTLD. Further study utilizing single cell transcriptomics would be of value in clearly identifying and validating this novel cell subset as well as help to understand what role such a cell type may play in disease pathogenesis.

The 35 biomarker genes identified as a classifier were not linked to obvious immune pathways. However, enrichment analysis with Enrichr did identify enrichment for several metabolomic pathways including glycine, serine, and threonine metabolism (BioPlanet30 and KEGG), lysine catabolismioPlanet) and glycolysis (MSigDB31). This is consistent with previous work identifying a metabolomic signature in PTLD20. In addition, a recent study identifying a set of 31 genes for diagnosis of acute Lyme disease also noted that only a fraction of the genes is immune related. These observations underscore the complex biology that is part of PTLD. Understanding the roles that immune and non-immune pathways play in the various stages of LD including PTLD will likely lead to novel therapeutic targets and other therapeutic strategies. The 35 biomarker genes are enriched in calcium ion channel regulation (CBARP, CACNB4) and are highly co-expressed with CAM kinases 2 and 4 (SLC4A10, CACNB4, NAP1L2, APBA1, PPFIA4, and SHANK1, marked in Table S3) all potentially forming a neuronal cell signaling pathway, further making a case for neurological symptoms.

This study produced a gene expression profile for PTLD. This is just a first step that requires confirmation for diagnosis of PTLD. Gene expression can support the diagnosis of PTLD in patients with a history of prior diagnosed and treated LD and persistent post-treatment symptoms. In addition, if a future diagnostic panel can suggest negative test results for PTLD, based on a reduced representation of gene expression profile, this could be valuable in patients with look-alike syndromes not associated with prior LD, and would lead to further evaluation of these patients to establish a definitive diagnosis.

The identified 35 biomarker genes may be useful as a diagnostic if the same approach is applied to whole blood instead of PBMCs. PBMC isolation is expensive and currently requires academic laboratory expertise. To translate the test into primary health care for LD patients, a parallel test will have to be devised by experimentally comparing PBMCs to whole blood results. This can be done computationally, but more reliably by experimentally measuring gene expression from PBMCs and whole blood from the same large cohort of diagnosed LD and PTLD patients.

### Examples

### Experimental Model And Subject Details

### Patient Cohorts

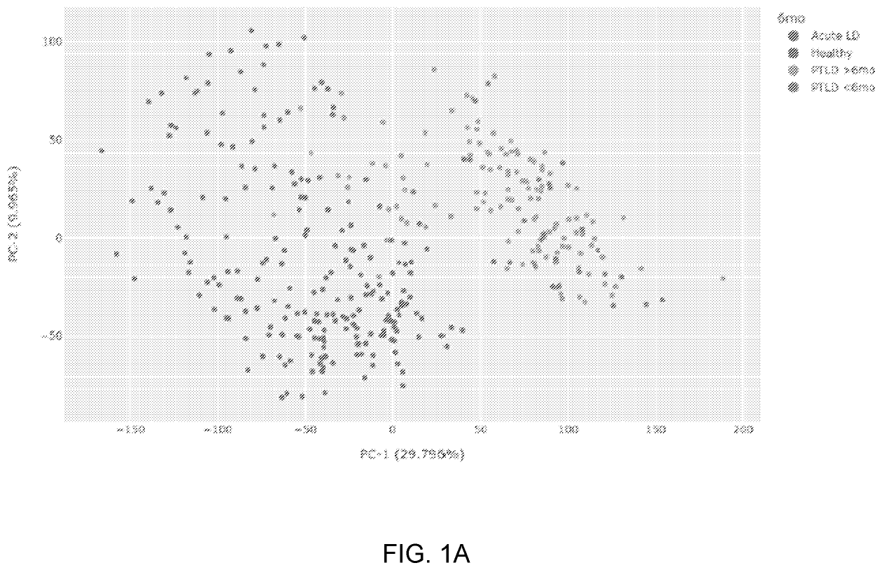

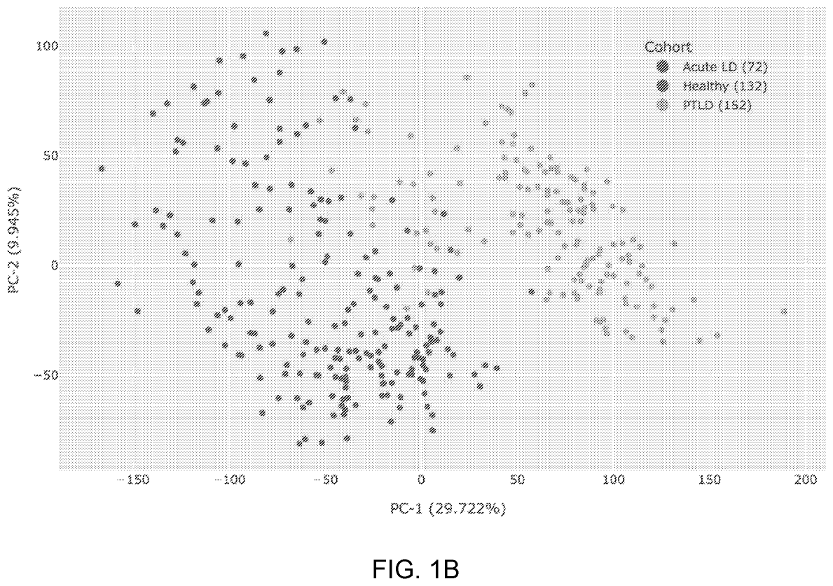

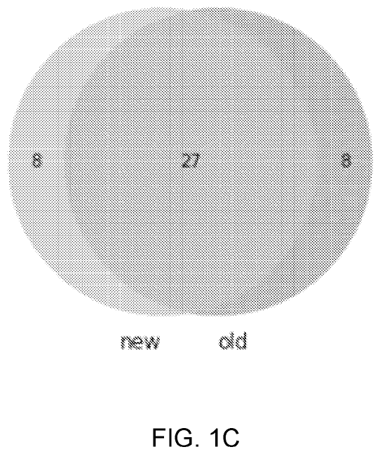

The cohorts of patients included in the current analysis are part of large, on-going studies to characterize patients with LD and PTLD. Samples from 152 patients with PTLD were drawn from a cross-sectional study for which detailed recruitment and eligibility information has been previously described. Briefly, participants were largely recruited from a clinic-based population during the period of 2008-2018 and were required to have medical record-confirmed prior LD and appropriate antibiotic treatment. Eligibility criteria included evidence of documented erythema migrans rash, oligoarthritis with joint swelling, facial palsy, neuropathy, meningitis, encephalitis, carditis, or a viral-like illness, as well as concurrent laboratory evidence of infection performed by a laboratory following CDC recommendations for test interpretation. Additionally, participants were required to have continued fatigue, pain, or cognitive dysfunction that affected function, and were excluded for a range of specific medical conditions with significant symptom overlap with PTLD. For the current analysis, it did not require participants to have been ill for at least 6 months at the time of enrollment. The implications of this decision were tested, and the makeup of the biomarker set was largely preserved even without considering the convalescent cohort patients. This is because these patients are uniformly mixed with the other PTLD patients (FIGS. 1A and 1C). Healthy controls were recruited from the same geographic region. They did not have a clinical history for LD and were CDC-negative on two-tier testing for antibodies to B. burgdorferi.

### Demographics of the Patient Cohort

The PTLD cohort consisted of 152 patients made of 66 females and 86 males. Their average age was 47.27 with a standard deviation of 15.85. Three patients in this cohort were self-identified as Asian, five as Hispanic, and three as Black while the remaining self-identified as White. The acute LD cohort consisted of 72 patients made of 31 females and 41 males. Their average age was 47.19 with a standard deviation of 15.68. One patient self-identified as Asian, and one as Black while the remaining self-identified as White. The healthy control cohort consisted of 45 patients made of 26 females and 19 males. Their average age was 50.29 with a standard deviation of 15.28. Five patients in this cohort were self-identified as Black, five as Hispanic, and one as Native American while the remaining self-identified as White.

### RNA-Seq Profiling of Patients

The RNA-seq profiles of participants with PTLD were compared to data from 72 patients with acute Lyme (‘acute LD’) who were then followed longitudinally up to one year after completing appropriate antibiotic treatment (convalescent cohort which was not considered in this study). Participants with acute LD had a physician-diagnosed EM rash present and ≤72 hours of appropriate antibiotic treatment at the time of enrollment. Finally, 44 healthy control participants without a clinical history of LD who were also two-tier seronegative for LD were also included. Additional details of the acute LD and control participants, as well as prior analysis of their RNA-seq profiles, were previously published.

The Institutional Review Board of the Johns Hopkins University School of Medicine approved this study, and all participants signed written consent prior to initiation of any study activities.

### Method Details

### Isolation of PBMC

PBMCs were isolated from fresh whole blood using Ficoll (Ficoll-Paque Plus, GE Healthcare) and total RNA was extracted from 107 PBMCs using RLT Lysis Buffer (Qiagen) by following manufacturer's instructions. The NEBNext Ultra II Directional RNA Library Prep Kit for Illumina (Cat #E7765) was used to generate RNA-seq libraries.

### Preparation of the Samples for RNA Sequencing

Poly A RNAs were isolated from total RNAs using NEBNext Poly(A) Magnetic Isolation Module (NEB #E7490) and then fragmented for cDNA synthesis. End repair is performed where 3′ to 5′ exonuclease activity of enzymes removes 3′ overhangs, and the polymerase activity fills in the 5′ overhangs. An ‘A’ base is then added to the 3′ end of the blunt phosphorylated DNA fragments which prepares the DNA fragments for ligation to the sequencing adapters, which have a single ‘T’ base overhang at their 3′ end. Ligated fragments are subsequently size selected through purification using the Sample Purification Beads included in the kit and undergo PCR amplification to prepare the ‘libraries. The BioAnalyzer is used for quality control of the libraries to ensure adequate concentration and appropriate fragment size free of adapter dimers. The resulting library insert size is 200 bp-500 bp with a median size around 300 bp. Libraries were barcoded and pooled for HiSeq2500 sequencing.

### RNA Sequencing

The prepared samples were processed by an Illumina HiSeq2500 sequencing instrument at the Genomics Core Facility at the Icahn School of Medicine at Mount Sinai.

### Quantification And Statistical Analysis

### RNA-Seq Processing

All samples taken from both studies were processed with FastQC, aligned to the human genome (gh38) with the STAR RNA-seq aligner33, after which Picard tools were used for gene, exon, and transcript quantification. The RNA-seq gene counts were merged, genes filtered by edgeR34 quasi-likelihood, log 2 transformed, quantile normalized and z-scored. Top differentially expressed genes were calculated using a limma-voom35,36 applied to the raw counts with a BH-corrected adjusted q-value cutoff of 0.01. Differentially expressed genes are computed by comparing pairwise between healthy controls, acute LD from patients at their first visit, and patients with PTLD.

### Enrichment Analysis

The differentially expressed genes were submitted to Enrichr13, an interactive web tool for performing Enrichment analysis. Each set was submitted independently, and a report of significant hits compiled.

### Set Overlap Analysis

The differentially expressed gene sets were further investigated using the SuperVenn package to visualize multi-set comparisons, helping to contrast gene sets against those in Enrichr. Enrichment analysis of GO Biological Processes using the consensus up-regulated genes between acute LD and PTLD revealed significant enrichment for cellular response to molecules of bacterial origin but also to inflammatory response. Consensus and divergent genes between the two groups which did not appear as biomarkers for inflammatory response, or several other viral or bacterial infections were considered as candidate biomarkers.

### Classification Model Construction and Candidate Biomarker Selection

The candidate biomarkers were further filtered by a variance selection criterion which scores biomarkers by total variance divided by inter-group variance and by permutation importance using Logistic Regression classifiers on four classification problems: LD vs. healthy, acute LD vs. healthy, PTLD vs. healthy, and acute LD vs. PTLD. The biomarkers achieving high scores for each of these categories were selected as features for the classification task. Additionally, the top single-gene biomarkers capable of separating samples were highlighted.

### Model Evaluation

To benchmark the generalizability of the approach, a third of the patients stratified across controls, acute LD, and PTLD were held out. Then, the same candidate biomarker selection approach was followed to produce a biomarker set. We constructed four independent pipelines consisting of scaling to unit mean and variance followed by a Logistic Regression classifier trained on the test set and evaluated using the held-out patients. To mitigate class imbalance, the test set was under sampled to have equal number of samples in the positive and negative classes during validation. The performance on the held-out set is visualized with Receiver Operating Characteristic (ROC) and Precision Recall (PR) curves, area under these curves (AUC) is computed and a confusion matrix produced from the true and false positives when considering a cutoff at 50% of the Logistic Regression Classifiers’ assigned probability. Additionally, permutation testing is applied on different train test splits to ensure results are consistent across many runs.

KEY RESOURCES TABLE

| | | |
|---|---|---|
| REAGENT or RESOURCE | SOURCE | IDENTIFIER |

Deposited data

| | | |
|---|---|---|
| PTLD RNA-seq FASTQ files | dbGAP | phs002797.v1.p1 |
| Acute LD RNA-seq FASTQ files | dbGAP | phs002795.v1.p1 |
| Processed data | Zenodo | 10.5281/zenodo.7084176 |

Software and algorithms

| | | |
|---|---|---|
| Deidentified processed RNA-seq data file | GitHub | N/A |
| https://github.com/LymeMIND/LM3-study-supporting-materials | | |
| Jupyter Notebook with code to reproduce the analysis | GitHub | N/A |
| https://github.com/LymeMIND/LM3-study-supporting-materials | | |
| Jupyter Notebook to reproduce figures | Zenodo | 10.5281/zenodo.7084176 |

### Results

RNA-seq profiles were collected from PBMCs isolated from 152 patients with PTLD. These patients were compared to previously published RNA-seq profiles from 72 patients with acute LD (acute cohort) and 44 healthy controls also followed over time as shown in Table 1 which is the distribution of RNA-seq patient samples across cohorts.

TABLE 1

| Study | Visit | | Cases | Controls |
|---|---|---|---|---|
| Acute LD Cohort | Baseline | | 72 | 44 |
| | 3 weeks | | 72 | N/A |
| | (end of treatment) | | | |
| Convalescent LD | 6 | months | 62 | 39 |
| Cohort (Not | | | | |
| considered in this | 1 | year | 61 | 25 |
| study for biomarker | 2 | years | N/A | 24 |

| | | | |
|---|---|---|---|
| development) | | | |
| PTLD Cohort | Any point after meeting | 152 | N/A |
| | criteria for PTLD | | |

Principal component analysis (PCA) of the dimensionality reduced profiles suggests that most patients with PTLD have an expression signature that is different from the healthy controls and profiled patients with acute LD (FIG. 1B). A smaller number of patients with PTLD show expression profiles that are comparable with those in the acute cluster.

To explore the characteristics of the patients with PTLD, they were compared to the healthy control and the acute LD groups. Differential expression analysis was followed by enrichment analysis.

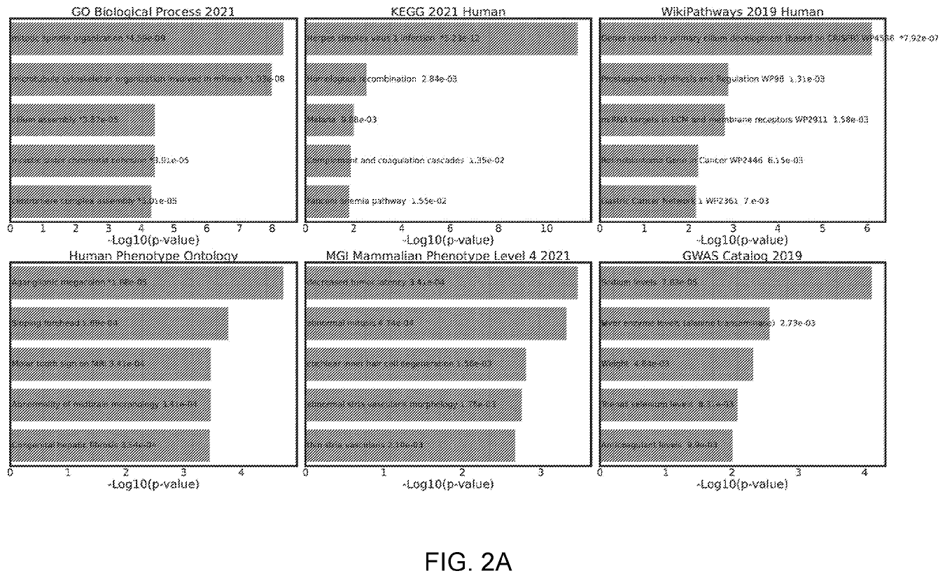

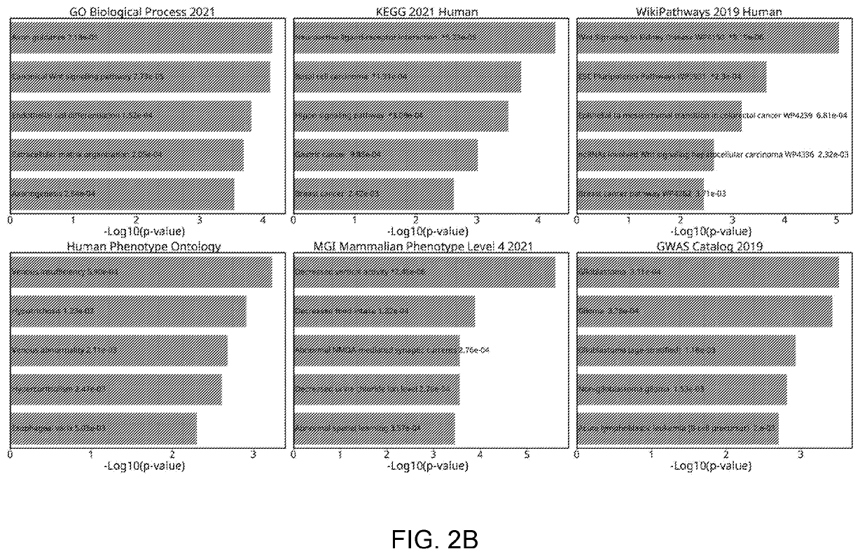

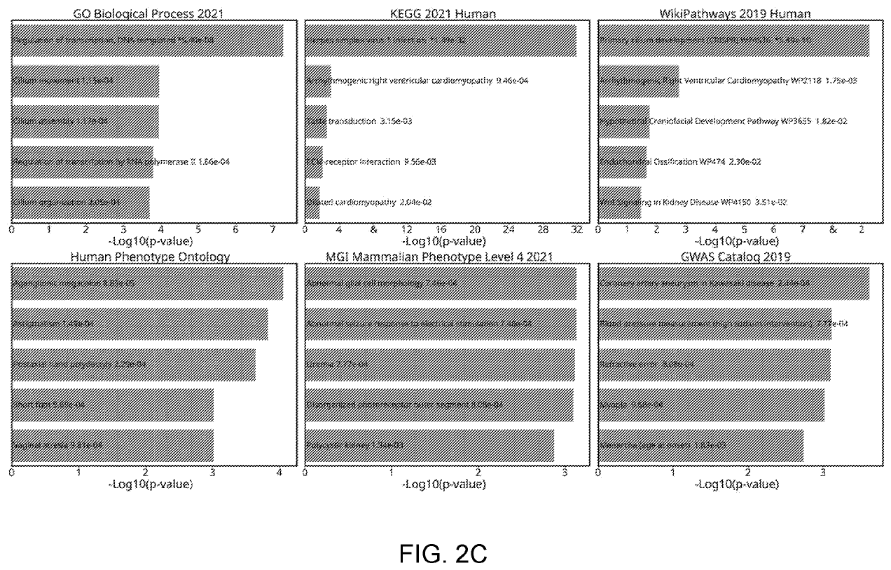

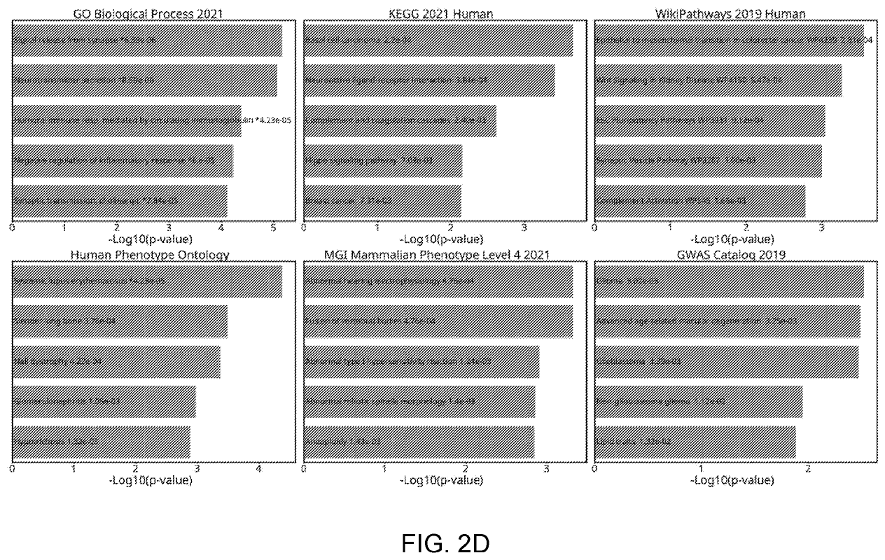

When comparing the patients with PTLD to healthy controls, 1213 genes were identified as significantly up-regulated in PTLD and 803 were identified as significantly down-regulated (limma-voom, BH-adjusted p-value <0.01). The enrichment results are presented as bar charts along with links to the reports in Enrichr (FIGS. 2A and 2B). The up-regulated genes are enriched for immune response genes and up-regulation of the cell cycle. Specifically, MSigDB Hallmark sets are enriched for G2-M checkpoint and E2F transcription factor targets (Fisher's exact test, p-value <0.000005; q-value <0.0001), and response to Herpes simplex virus 1 infection (KEGG pathways, p-value <5.3e-12; q-value <1.54e-9). Interestingly, a significant number of differentially expressed genes are enriched for cilium components (GO (GO: 0005929), p-value <0.0006; q-value <0.1) and cilium related disorders (primary ciliary dyskinesia, p-value <0.00008). Down regulated genes, when comparing patients with PTLD to healthy controls are enriched for Wnt pathway components (Wiki Pathways, Wnt signaling in kidney disease WP4150, p-value <0.00001; q-value <0.003), and spinal cord specification genes (Tissue Protein Expression from Human Proteome Map, adult spinal cord, p-value <0.00009; q-value <0.0027).

When comparing the 152 gene expression profiles from patients with PTLD to the 72 patients with acute LD, similar patterns were observed (FIGS. 2C and 2D). Specifically, 817 genes are significantly up-regulated in the patients with PTLD, and these genes are enriched for activation of the cell cycle compared to the patients with acute LD, for example, “Metaphase/Anaphase Phase Transition” from the Elsevier Pathway Collection (p-value <0.000006; q-value <0.0022). Interestingly, the same gene sets that are up-regulated when comparing the PTLD group to healthy controls are down-regulated when comparing PTLD to acute LD (KEGG, Herpes simplex virus 1 infection, p-value <1.5e-32; q-value <4.39e-30) (FIG. 3).

After processing this data to identify differentially expressed genes, the results were analyzed and visualized with a SuperVenn diagram (FIG. 4). The differentially expressed genes from the acute LD and PTLD cohorts were compared with gene sets from other infectious diseases (Table 2). The additional gene sets were extracted from Enrichr13. Such gene sets include gene sets extracted from human PBMCs virally infected with Influenza, HIV, and COVID-19 published studies. These gene sets were filtered by genes which are also differentially expressed in acute LD or PTLD. Bacterial infection response genes derived from Enrichr gene sets are those corresponding to studies that profiled human PBMCs from patients infected with pathogens such as Mycobacterium tuberculosis, and Neisseria gonorrhoeae. In addition, gene sets related to Streptococcus pneumoniae, Staphylococcus aureus, Escherichia coli, and Bacterial sepsis extracted from studies that profiled human cells infected with those bacteria were included. Also, gene sets from DisGeNET14, and those corresponding to diseases caused specifically by Spirochetes filtered by genes which are also differentially expressed in acute LD or PTLD were included (Table 2).

TABLE 2
Data Sources for Gene Sets

| Category | Disease | Enrichr Library | Term | Genes |
|---|---|---|---|---|
| Term | | GO_Biological_Process_{—} | inflammatory response (GO: 0006954) | 253 |
| | | 2018 | | |
| Term | | GO_Biological_Process_{—} | cellular response to molecule of | 85 |
| | | 2018 | bacterial origin (GO: 0071219) | |
| Viral | Influenza | Microbe_Perturbations_{—} | H1N1 influenza virus (pandemic | 447 |
| | | from_GEO_up | strain) human peripheral blood cells | |
| | | | GDS4240 microbe: 174 | |
| Viral | Influenza | Microbe_Perturbations_{—} | H1N1 influenza virus (pandemic | 405 |
| | | from_GEO_up | strain) human peripheral blood cells | |
| | | | GDS4240 microbe: 173 | |
| Viral | HIV | Microbe_Perturbations_{—} | HIV-1 human PBMC GDS1449 | 374 |
| | | from_GEO_up | microbe: 221 | |
| Viral | COVID-19 | COVID- | COVID-19 patients PBMC up | 707 |
| | | 19_Related_Gene_Sets | | |
| Bacterial | Tuberculosis | Microbe_Perturbations_{—} | Mycobacterium tuberculosis human | 275 |
| | | from_GEO_up | PBMC GDS4966 microbe: 223 | |
| Bacterial | Tuberculosis | Microbe_Perturbations_{—} | Mycobacterium tuberculosis human | 358 |
| | | from_GEO_up | PBMC GSE16250 microbe: 237 | |
| Bacterial | Streptococcus | Microbe_Perturbations_{—} | Streptococcus pneumoniae G54 | 176 |
| | | from_GEO_up | human pharyngeal epithelial cells | |
| | | | GDS3041 microbe: 16 | |
| Bacterial | Streptococcus | Microbe_Perturbations_{—} | Streptococcus pneumoniae G54 | 309 |
| | | from_GEO_up | human pharyngeal epithelial cells | |
| | | | GDS3041 microbe: 19 | |
| Bacterial | Streptococcus | Microbe_Perturbations_{—} | Streptococcus pneumoniae TIGR4 | 318 |
| | | from_GEO_up | human pharyngeal epithelial cells | |
| | | | GDS3041 microbe: 17 | |
| Bacterial | Streptococcus | Microbe_Perturbations_{—} | Streptococcus pneumoniae D39 | 140 |
| | | from_GEO_up | human pharyngeal epithelial cells | |
| | | | GDS3041 microbe: 14 | |
| Bacterial | Streptococcus | Microbe_Perturbations_{—} | Streptococcus pneumoniae D39 | 242 |
| | | from_GEO_up | human pharyngeal epithelial cells | |
| | | | GDS3041 microbe: 18 | |
| Bacterial | Streptococcus | Microbe_Perturbations_{—} | Streptococcus pneumoniae TIGR4 | 362 |
| | | from_GEO_up | human pharyngeal epithelial cells | |
| | | | GDS3041 microbe: 20 | |
| Bacterial | Streptococcus | Microbe_Perturbations_{—} | Streptococcus pneumoniae D39 | 222 |
| | | from_GEO_up | human pharyngeal epithelial cells | |
| | | | GDS3041 microbe: 15 | |
| Bacterial | Pneumococcal | DisGeNET | Meningitis, Pneumococcal | 28 |
| Bacterial | Pneumococcal | DisGeNET | Pneumococcal Infections | 43 |
| Bacterial | E. Coli | Microbe_Perturbations_{—} | Escherichia coli mouse bladder | 291 |
| | | from_GEO_up | urothelium GDS2977 microbe: 75 | |
| Bacterial | E. Coli | Microbe_Perturbations_{—} | Uropathogenic Escherichia coli | 240 |
| | | from_GEO_up | mouse urothelium, distal GSE6419 | |
| | | | microbe: 340 | |
| Bacterial | E. Coli | Microbe_Perturbations_{—} | Uropathogenic Escherichia coli | 315 |
| | | from_GEO_up | mouse urothelium, proximal | |
| | | | GSE6419 microbe: 341 | |
| Bacterial | E. Coli | Microbe_Perturbations_{—} | Escherichia coli mouse bladder | 224 |
| | | from_GEO_up | urothelium GDS2977 microbe: 76 | |
| Bacterial | E. Coli | Disease_Perturbations_{—} | Escherichia coli infection of the | 375 |
| | | from_GEO_up | central nervous system C1320188 | |
| | | | mouse GSE3253 sample 341 | |
| Bacterial | Staphylococcus | Microbe_Perturbations_{—} | Staphylococcus aureus human | 320 |
| | | from_GEO_up | macrophage GDS4931 microbe: 60 | |
| Bacterial | Staphylococcus | Microbe_Perturbations_{—} | Staphylococcus aureus human | 319 |
| | | from_GEO_up | monocyte-derived macrophages | |
| | | | GDS4931 microbe: 61 | |
| Bacterial | Staphylococcus | Microbe_Perturbations_{—} | Staphylococcus aureus human | 226 |
| | | from_GEO_up | bronchial epithelial BEAS-2B cells | |
| | | | GDS2606 microbe: 135 | |
| Bacterial | Staphylococcus | Microbe_Perturbations_{—} | Staphylococcus aureus human | 352 |
| | | from_GEO_up | monocyte-derived macrophages | |
| | | | GDS4931 microbe: 62 | |
| Bacterial | Gonorrhoeae | Microbe_Perturbations_{—} | Neisseria gonorrhoeae human | 413 |
| | | from_GEO_up | peripheral blood mononuclear cells | |
| | | | (PBMC) GDS1646 microbe: 171 | |
| Bacterial | Gonorrhoeae | Microbe_Perturbations_{—} | Neisseria gonorrhoeae human | 352 |
| | | from_GEO_up | peripheral blood mononuclear cells | |
| | | | (PBMC) GDS1646 microbe: 172 | |
| Bacterial | Sepsis | Disease_Perturbations_{—} | Sepsis C0243026 mouse GSE4479 | 284 |
| | | from_GEO_up | sample 150 | |
| Bacterial | Sepsis | Disease_Perturbations_{—} | Sepsis C0243026 rat GSE1781 | 226 |
| | | from_GEO_up | sample 314 | |
| Bacterial | Sepsis | DisGeNET | Sepsis of the newborn | 6 |
| Bacterial | Sepsis | DisGeNET | Sepsis due to urinary tract infection | 5 |
| Bacterial | Sepsis | DisGeNET | Abdominal sepsis | 6 |
| Bacterial | Sepsis | DisGeNET | Severe Sepsis | 102 |
| Bacterial | Sepsis | DisGeNET | Neonatal Early-Onset Sepsis | 5 |
| Bacterial | Sepsis | DisGeNET | Sepsis | 528 |
| Bacterial | Sepsis | DisGeNET | Bacterial sepsis of newborn | 5 |
| Bacterial | Sepsis | DisGeNET | Bacterial sepsis | 14 |
| Spirochetes | Syphilis | DisGeNET | Syphilis | 20 |
| Spirochetes | Syphilis | Jensen_DISEASES | Syphilis | 21 |
| Spirochetes | Leptospirosis | DisGeNET | Leptospirosis | 14 |
| Spirochetes | Leptospirosis | Jensen_DISEASES | Leptospirosis | 15 |
| Spirochetes | Yaws | Jensen_DISEASES | Yaws | 7 |
| Spirochetes | Relapsing Fever | DisGeNET | Relapsing Fever | 12 |
| Spirochetes | Relapsing Fever | Jensen_DISEASES | Relapsing fever | 13 |
| Spirochetes | Periodontal | DisGeNET | Periodontal Diseases | 136 |
| | Disease | | | |
| Spirochetes | Bejel | Rare_Diseases_GeneRIF_{—} | Bejel | 23 |
| | | Gene_Lists | | |
| Spirochetes | Bejel | Rare_Diseases_AutoRIF_{—} | Bejel | 119 |
| | | Gene_Lists | | |
| Spirochetes | Pinta | Rare_Diseases_AutoRIF_{—} | Pinta | 53 |
| | | Gene_Lists | | |

Next, processing the Super Venn diagram for overlapping sets, the differentially expressed genes from the acute LD and the PTLD groups that were specifically not present in any viral or bacterial infection gene sets were extracted. The resulting gene set contained 41 shared LD up genes, 134 shared LD down genes, and 175 genes which appear to be affected in opposite directions between acute LD and PTLD. The set of 350 candidate genes were further reduced using permutation importance with the goal of achieving an optimal tradeoff between performance and gene set size. Permutation importance was calculated with Logistic Regression classifiers trained on randomly selected 50% of the samples to predict the class labels of the remaining 50%. Furthermore, the permutation importance was performed using four separate binary classification tasks: a) distinguishing acute LD and PTLD together from healthy controls; b) distinguishing acute LD from healthy controls; c) distinguishing PTLD from healthy controls; and d) distinguishing between healthy controls and acute LD or PTLD.

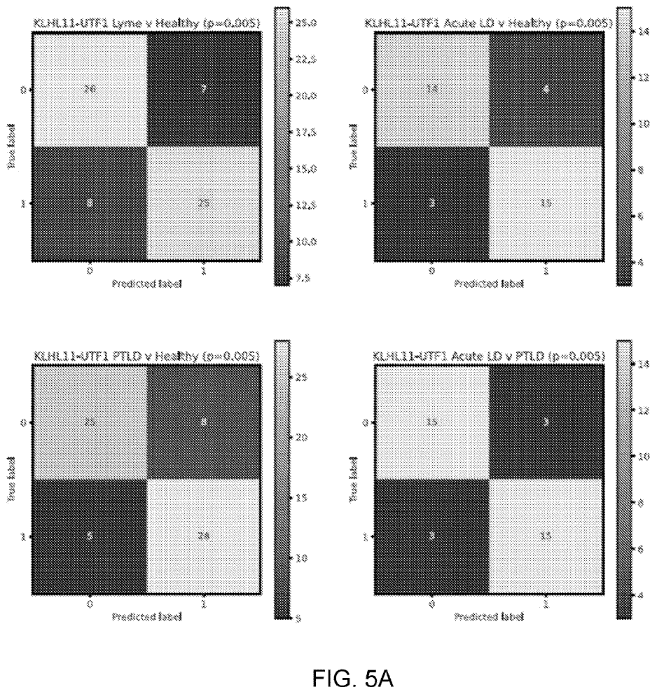

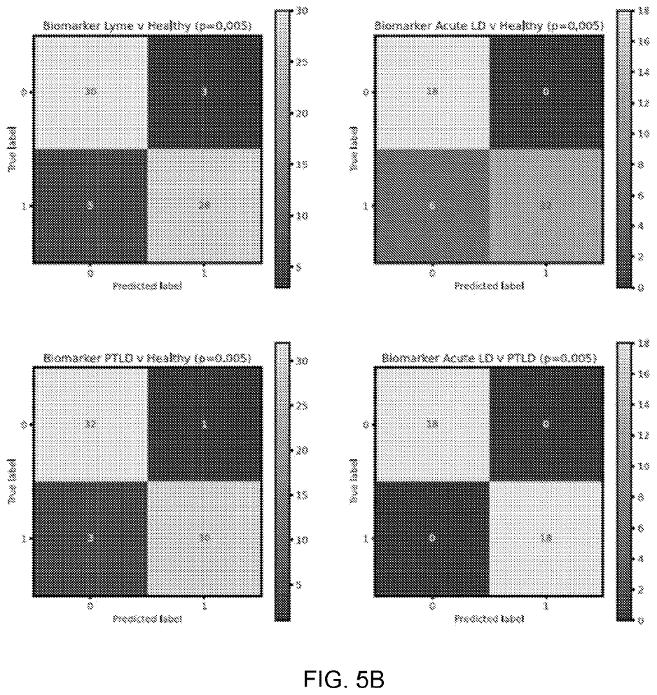

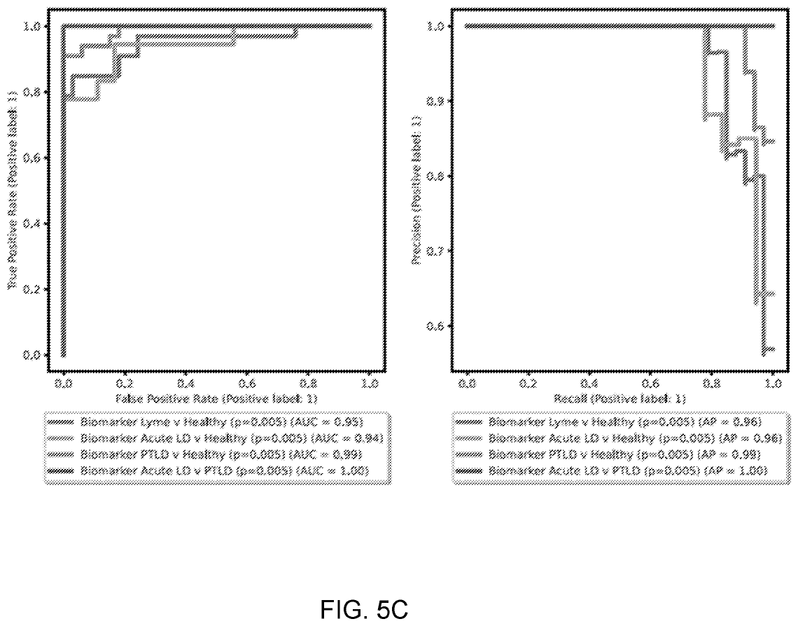

The top k genes from the gene permutation importance test can be used to train a classifier which performs increasingly well as k becomes larger. It was found that at around 35 genes, where a split in the permutation importance distribution occurs, one only loses 0.04 ROC AUC for the acute LD vs. healthy control classifier when compared to using all 350 genes. At fewer than 35 genes, performance begins to degrade rapidly. With 35 genes (Table 3), the classifier performs at 98% accuracy determining whether a sample is from a LD patient or a healthy control (FIG. 5).

TABLE 3
The 35 mRNA biomarker genes for distinguishing healthy controls,
acute LD, and PTLD.

| Gene | LD v | acute LD v | PTLD v | acute LD v | Mean (total_var / | |
|---|---|---|---|---|---|---|
| Symbol | healthy | healthy | healthy | PTLD | inter_group_var) | Score |
| KLAL11 | 17.58 | 17.59 | 13.74 | 1.55 | 0.05 | 10.1 |
| UTF1 | −0.13 | −0.11 | −0.03 | 17.15 | 4.84 | 4.34 |
| NBPF1 | −0.13 | 4.09 | 6.23 | −0.05 | 0.32 | 2.09 |
| RBMS3 | −0.13 | −0.11 | −0.13 | −0.11 | 10.16 | 1.94 |
| PPFIA4* | 0.45 | −0.11 | 9.39 | −0.11 | 0.04 | 1.94 |
| NOTCH3 | −0.05 | 0.02 | −0.08 | −0.11 | 9.51 | 1.86 |
| SLC4A10* | 1.95 | 2.03 | 2.71 | −0.11 | 0.5 | 1.42 |
| TMEM272 | 3.12 | −0.11 | 3.51 | −0.11 | 0.15 | 1.31 |
| CBARP* | −0.13 | −0.11 | −0.13 | 0.12 | 5.68 | 1.09 |
| MLANA | 0.27 | −0.11 | 0.46 | 3.84 | −0.19 | 0.85 |
| CHDH | 0.31 | 2.89 | −0.13 | −0.11 | 1.25 | 0.84 |
| NAP1L2* | −0.13 | −0.03 | −0.13 | 4.18 | 0.03 | 0.78 |
| TMEM52B | −0.13 | 0.12 | −0.13 | −0.11 | 4.04 | 0.76 |
| C2CD4D | 1.35 | 0.15 | 0.53 | 0.07 | 1.47 | 0.71 |
| OTUB2 | −0.13 | −0.05 | 0.01 | 3.84 | −0.11 | 0.71 |
| POMK | 0.56 | −0.11 | 2.45 | 0.07 | 0.4 | 0.67 |
| KCNG1 | −0.05 | 1.96 | 1.12 | −0.11 | 0.37 | 0.66 |
| CAND2 | 2.22 | 0 | −0.1 | 0.01 | 0.24 | 0.47 |
| DCSTAMP | −0.13 | −0.11 | −0.13 | −0.11 | 2.8 | 0.47 |
| NBEALI | 1.38 | 0.43 | −0.13 | −0.11 | 0.73 | 0.46 |
| SCN3A | 1.65 | 0.03 | 0.23 | 0.12 | 0.17 | 0.44 |
| AHNAK2 | 1.49 | 0.93 | −0.13 | −0.11 | −0.13 | 0.41 |
| RAD50 | 1.4 | 0.54 | −0.13 | −0.11 | 0.02 | 0.35 |
| APBA1* | 1.47 | 0.36 | −0.1 | −0.11 | −0.14 | 0.3 |
| DNAH7 | −0.13 | −0.11 | −0.13 | −0.11 | 1.71 | 0.25 |
| CXADR | −0.13 | −0.11 | −0.13 | −0.11 | 1.6 | 0.23 |
| AMH | −0.12 | −0.03 | −0.13 | −0.11 | 1.42 | 0.21 |
| ALDH7A1 | −0.13 | 0.03 | −0.13 | −0.11 | 1.37 | 0.21 |
| CACNB4* | −0.13 | −0.11 | −0.13 | −0.11 | 1.48 | 0.2 |
| CMTM1 | −0.11 | 0.14 | −0.13 | −0.11 | 1.19 | 0.2 |
| SHANK1* | −0.13 | −0.11 | −0.08 | −0.11 | 1.4 | 0.19 |
| TULP2 | 0.3 | 0.44 | 0.58 | 0.12 | −0.48 | 0.19 |
| NEKS | −0.13 | −0.11 | −0.13 | −0.11 | 1.43 | 0.19 |
| BTNL9 | 0.32 | 0.24 | 0.09 | −0.11 | 0.39 | 0.19 |
| GPR135 | 1.11 | −0.01 | −0.13 | −0.11 | 0 | 0.17 |

In Table 3 average feature importance score based on normalized feature importance for classifiers trained to distinguish classes pairwise along with a normalized measure of maximal extra-group variance and minimal inter-group variance. Top 3 performers in each category are bolded. A set of co-expressed genes associated with synaptic calcium signaling are marked with asterisk.

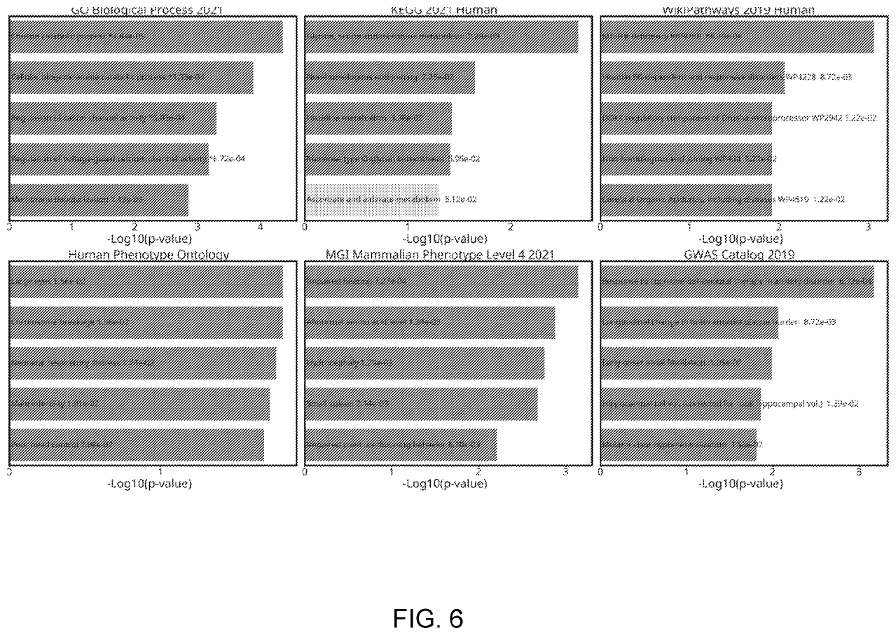

Since the 35 selected biomarker genes are specific to LD and are not known to be associated with immune activation functions, it was attempted to explore enriched terms associated with these 35 genes (FIG. 6). Interestingly, the genes CACNB4 and ALDH7A1 are highly enriched for epilepsy based on OMIM15 (Fisher exact test, p-value <0.0047, q-value <0.0093), and the genes CACNB4, ALDH7A1, SLC4A10, and SCN3A are enriched for proteins involved in epilepsy based on the Elsevier Pathway Collection database (p-value <0.0013, q-value <0.093). Neuropsychiatric symptoms have been reported in patients with PTLD and in patients with Lyme encephalopathy 16,17 and this analysis suggests a molecular underpinning of such a phenotype. Three of the genes from the set of 35 biomarkers, namely AHNAK2, APBA1 and SHANK1, encode genes with a PDZ domain which is statistically over-represented (p-value <0.0015, q-value <0.034). These genes may form complexes with the several ion channels that are over-represented in the list of 35 biomarkers and potentially impact the neurologic symptoms observed in PTLD. Applying WEAT analysis18, it was observed that most of the 35 have some annotations, but they are mostly under-studied (Table 4).

TABLE 4
WEAT analysis. The submitted gene set is the 35-biomarker set with
the settings of “Importance/GIC” and scaling factor 3.

| Library | Counts | All | Coverage (%) |
|---|---|---|---|
| KEGG | 24 | 14841 | 68.57 |
| WikiPathways_Human | 7 | 6201 | 20 |
| BioCarta | 1 | 1348 | 2.86 |
| GO_cellular_component | 33 | 18903 | 94.29 |
| GO_molecular_function | 33 | 18189 | 94.29 |
| GO_biological_process | 31 | 17968 | 88.57 |
| Human_Phenotype_Ontology_With_Links | 6 | 3096 | 17.14 |
| Reactome | 12 | 8973 | 34.29 |
| OMIM_Expanded | 2 | 2178 | 5.71 |
| Genome_Browser_PWMs | 18 | 13362 | 51.43 |
| Ligand_Perturbations_from_GEO_merged | 24 | 19772 | 68.57 |
| Cancer_Cell_Line_Encyclopedia | 23 | 15797 | 65.71 |
| NIH_Funded_PIs_2017_Human_GeneRIF | 22 | 13072 | 62.86 |
| Gene_Perturbations_from_GEO_down | 31 | 31132 | 88.57 |
| Elsevier_Pathway_Collection | 12 | 5718 | 34.29 |
| ENCODE_TF_ChIP-seq_2015 | 31 | 26382 | 88.57 |
| InterPro_Domains_2019 | 25 | 12444 | 71.43 |
| Disease_Signatures_from_GEO_2014_merged | 28 | 18007 | 80 |
| Enrichr_Libraries_Most_Popular_Genes | 7 | 5902 | 20 |
| MGI_Mammalian_Phenotype_2017 | 16 | 8184 | 45.71 |
| HomoloGene | 32 | 19129 | 91.43 |
| ARCHS4_Cell-lines | 34 | 23601 | 97.14 |
| L1000_Kinase_and_GPCR_Perturbations_merged | 23 | 12719 | 65.71 |
| Virus_Perturbations_from_GEO_down | 27 | 17576 | 77.14 |
| Data_Acquisition_Method_Most_Popular_Genes | 1 | 1073 | 2.86 |
| Gene_Perturbations_from_GEO_merged | 32 | 33616 | 91.43 |
| L1000_Kinase_and_GPCR_Perturbations_up | 23 | 12638 | 65.71 |
| MGI_Mammalian_Phenotype_Level_3 | 10 | 10406 | 28.57 |
| HumanCyc_2016 | 2 | 934 | 5.71 |
| TRRUST_Transcription_Factors_2019 | 6 | 3264 | 17.14 |
| OMIM_Disease | 2 | 1759 | 5.71 |
| VirusMINT | 2 | 851 | 5.71 |
| Drug_Perturbations_from_GEO_2014 | 31 | 47107 | 88.57 |
| huMAP | 2 | 2243 | 5.71 |
| NIH_Funded_PIs_2017_Human_AutoRIF | 28 | 13464 | 80 |
| Pfam_Domains_2019 | 19 | 9000 | 54.29 |
| NCI-Nature_2016 | 3 | 2541 | 8.57 |
| Disease_Signatures_from_GEO_2014_up | 25 | 15057 | 71.43 |
| Achilles_fitness_decrease | 6 | 4271 | 17.14 |
| GTEx_Tissue_Sample_Gene_Expression_Profiles_up | 3 | 4360 | 8.57 |
| Jensen_COMPARTMENTS | 27 | 18329 | 77.14 |
| GeneSigDB | 30 | 23726 | 85.71 |
| Rare_Diseases_GeneRIF_ARCHS4_Predictions | 25 | 13929 | 71.43 |
| Microbe_Perturbations_from_GEO_down | 18 | 15854 | 51.43 |
| Disease_Perturbations_from_GEO_down | 32 | 23939 | 91.43 |
| ENCODE_Histone_Modifications_2015 | 32 | 29065 | 91.43 |
| Rare_Diseases_GeneRIF_Gene_Lists | 20 | 10352 | 57.14 |
| DrugMatrix | 6 | 5209 | 17.14 |
| Pfam_InterPro_Domains | 13 | 7588 | 37.14 |
| TG_GATES_2020 | 18 | 12118 | 51.43 |
| Achilles_fitness_increase | 7 | 4320 | 20 |
| Old_CMAP_down | 17 | 8695 | 48.57 |
| GWAS_Catalog_2019 | 18 | 19378 | 51.43 |
| HMS_LINCS_KinomeScan | 1 | 392 | 2.86 |
| TargetScan_microRNA | 8 | 7504 | 22.86 |
| Table_Mining_of_CRISPR_Studies | 21 | 14156 | 60 |
| ProteomicsDB_2020 | 11 | 8189 | 31.43 |
| L1000_Kinase_and_GPCR_Perturbations_down | 23 | 12668 | 65.71 |
| Gene_Perturbations_from_GEO_up | 32 | 30832 | 91.43 |
| HMDB_Metabolites | 6 | 3723 | 17.14 |
| Allen_Brain_Atlas_merged | 21 | 13956 | 60 |
| MSigDB_Computational | 14 | 10061 | 40 |
| TRANSFAC_and_JASPAR_PWMs | 30 | 27884 | 85.71 |
| TF-LOF_Expression_from_GEO | 30 | 34061 | 85.71 |
| MSigDB_Hallmark_2020 | 7 | 4383 | 20 |
| Disease_Perturbations_from_GEO_merged | 32 | 27817 | 91.43 |
| Old_CMAP_merged | 22 | 12316 | 62.86 |
| BioPlanet_2019 | 12 | 9813 | 34.29 |
| Disease_Perturbations_from_GEO_up | 31 | 23561 | 88.57 |
| Epigenomics_Roadmap_HM_ChIP-seq | 30 | 22288 | 85.71 |
| TargetScan_microRNA_2017 | 33 | 17598 | 94.29 |
| DepMap_WG_CRISPR_Screens_Sanger_CellLines_20 | 8 | 6204 | 22.86 |
| Allen_Brain_Atlas_down | 21 | 13877 | 60 |
| PPI_Hub_Proteins | 13 | 16399 | 37.14 |
| PheWeb_2019 | 15 | 9116 | 42.86 |
| dbGaP | 10 | 5613 | 28.57 |
| LINCS_L1000_Ligand_Perturbations_down | 7 | 3788 | 20 |
| ARCHS4_IDG_Coexp | 31 | 20883 | 88.57 |
| Rare_Diseases_AutoRIF_ARCHS4_Predictions | 25 | 13787 | 71.43 |
| Aging_Perturbations_from_GEO_down | 22 | 16129 | 62.86 |
| Genes_Associated_with_NIH_Grants | 24 | 15886 | 68.57 |
| MCF7_Perturbations_from_GEO_down | 20 | 15022 | 57.14 |
| TF_Perturbations_Followed_by_Expression | 31 | 19741 | 88.57 |
| Virus_Perturbations_from_GEO_merged | 31 | 19391 | 88.57 |
| Human_Phenotype_Ontology | 6 | 3096 | 17.14 |
| MSigDB_Oncogenic_Signatures | 23 | 11250 | 65.71 |
| COVID-19_Related_Gene_Sets | 23 | 16979 | 65.71 |
| LINCS_L1000_Chem_Pert_down | 16 | 9448 | 45.71 |
| Kinase_Perturbations_from_GEO_merged | 30 | 19789 | 85.71 |
| Rare_Diseases_AutoRIF_Gene_Lists | 21 | 10471 | 60 |
| ARCHS4_TFs_Coexp | 34 | 25983 | 97.14 |
| Disease_Signatures_from_GEO_2014_down | 22 | 15406 | 62.86 |
| MCF7_Perturbations_from_GEO_merged | 25 | 19135 | 71.43 |
| Human_Gene_Atlas | 23 | 13373 | 65.71 |
| DepMap_WG_CRISPR_Screens_Broad_CellLines_201 | 11 | 7744 | 31.43 |
| Kinase_Perturbations_from_GEO_down | 28 | 17850 | 80 |
| GTEx_Tissue_Sample_Gene_Expression_Profiles_do | 28 | 16725 | 80 |
| MCF7_Perturbations_from_GEO_up | 20 | 15676 | 57.14 |
| CORUM | 4 | 2741 | 11.43 |
| Ligand_Perturbations_from_GEO_down | 17 | 15090 | 48.57 |
| MGI_Mammalian_Phenotype_Level_4 | 11 | 10493 | 31.43 |
| Old_CMAP_up | 22 | 11251 | 62.86 |
| Microbe_Perturbations_from_GEO_merged | 22 | 20108 | 62.86 |
| Mouse_Gene_Atlas | 26 | 19270 | 74.29 |
| Chromosome_Location_hg19 | 34 | 27360 | 97.14 |
| NIH_Funded_PIs_2017_GeneRIF_ARCHS4_Prediction | 28 | 17258 | 80 |
| Aging_Perturbations_from_GEO_up | 21 | 15309 | 60 |
| miRTarBase_2017 | 29 | 14893 | 82.86 |
| ESCAPE | 28 | 25648 | 80 |
| GTEx_Tissue_Sample_Gene_Expression_Profiles_me | 30 | 17315 | 85.71 |
| NCI-60_Cancer_Cell_Lines | 22 | 12232 | 62.86 |
| Ligand_Perturbations_from_GEO_up | 17 | 15103 | 48.57 |
| knockTF | 30 | 21755 | 85.71 |
| Drug_Perturbations_from_GEO_down | 30 | 23877 | 85.71 |
| Panther_2016 | 5 | 2041 | 14.29 |
| ENCODE_and_ChEA_Consensus_TFs_from_ChIP-X | 25 | 15562 | 71.43 |
| LINCS_L1000_Chem_Pert_merged | 18 | 10330 | 51.43 |
| ARCHS4_Tissues | 33 | 21809 | 94.29 |
| Tissue_Protein_Expression_from_Human_Proteome_{—} | 16 | 6454 | 45.71 |
| Kinase_Perturbations_from_GEO_up | 28 | 17660 | 80 |
| KEA_2015 | 5 | 3101 | 14.29 |
| Transcription_Factor_PPIs | 10 | 6002 | 28.57 |
| DSigDB | 32 | 19512 | 91.43 |
| Aging_Perturbations_from_GEO_merged | 27 | 19817 | 77.14 |
| Phosphatase_Substrates_from_DEPOD | 0 | 280 | 0 |
| NURSA_Human_Endogenous_Complexome | 14 | 10231 | 40 |
| knockTF_Top20 | 16 | 5778 | 45.71 |
| ClinVar_2019 | 1 | 1397 | 2.86 |
| BioPlex_2017 | 17 | 10271 | 48.57 |
| Tissue_Protein_Expression_from_ProteomicsDB | 27 | 13572 | 77.14 |
| Chromosome_Location | 28 | 32740 | 80 |
| Jensen_DISEASES | 25 | 15755 | 71.43 |
| HumanCyc_2015 | 2 | 756 | 5.71 |
| Virus-Host_PPI_P-HIPSTer_2020 | 12 | 5605 | 34.29 |
| LINCS_L1000_Chem_Pert_up | 18 | 9559 | 51.43 |
| SILAC_Phosphoproteomics | 6 | 5655 | 17.14 |
| ARCHS4_Kinases_Coexp | 28 | 19612 | 80 |
| DisGeNET | 28 | 17464 | 80 |
| UK_Biobank_GWAS_v1 | 24 | 14147 | 68.57 |
| ChEA_2016 | 32 | 49238 | 91.43 |
| MGI_Mammalian_Phenotype_Level_4_2019 | 19 | 13420 | 54.29 |
| NIH_Funded_PIs_2017_AutoRIF_ARCHS4_Predictions | 29 | 16964 | 82.86 |
| Enrichr_Users_Contributed_Lists_2020 | 35 | 54967 | 100 |
| Jensen_TISSUES | 30 | 19586 | 85.71 |
| Drug_Perturbations_from_GEO_up | 30 | 24350 | 85.71 |
| Allen_Brain_Atlas_up | 20 | 13121 | 57.14 |
| Microbe_Perturbations_from_GEO_up | 17 | 15015 | 48.57 |
| CCLE_Proteomics_2020 | 17 | 11851 | 48.57 |
| lncHUB_lncRNA_Co-Expression | 35 | 18704 | 100 |
| Virus_Perturbations_from_GEO_up | 29 | 17711 | 82.86 |
| SubCell_BarCode | 21 | 12419 | 60 |
| SysMyo_Muscle_Gene_Sets | 26 | 19500 | 74.29 |
| RNA-Seq_Disease_Gene_and_Drug_Signatures_from | 30 | 22440 | 85.71 |
| LINCS_L1000_Ligand_Perturbations_merged | 7 | 4711 | 20 |
| ENCODE_TF_ChIP-seq_2014 | 28 | 21493 | 80 |
| Drug_Perturbations_from_GEO_merged | 32 | 27853 | 91.43 |
| LINCS_L1000_Ligand_Perturbations_up | 5 | 3357 | 14.29 |
| Enrichr_Submissions_TF-Gene_Coocurrence | 25 | 12486 | 71.43 |

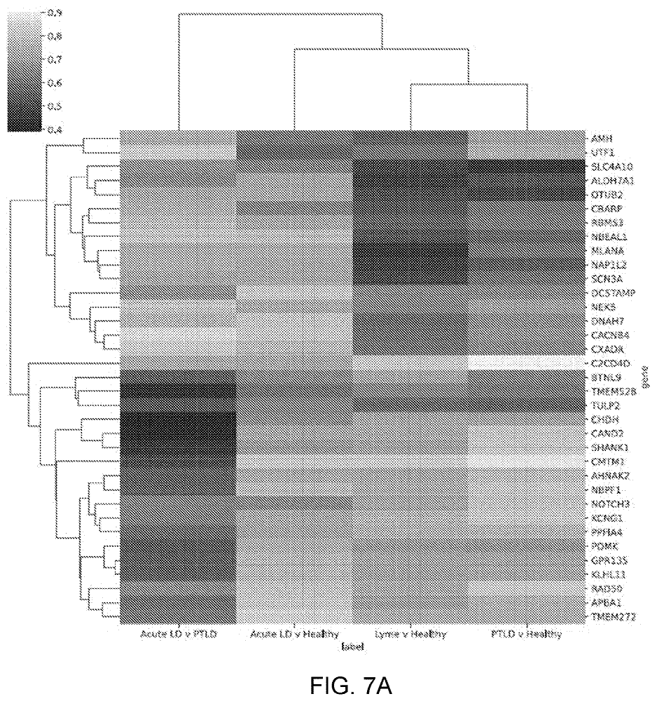

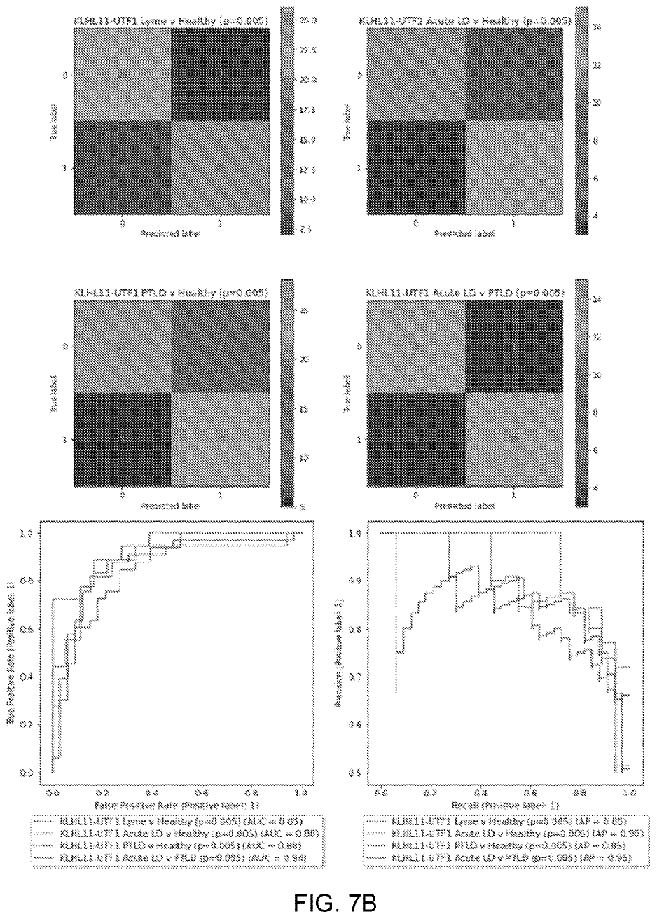

Nonetheless, due to the strong alignment of the samples to the two principal components, it was sought to identify the most singularly predictive genes to find an adequate proxy for the principal components. The top ranked gene based on the permutation importance analysis is Kelch Like Family Member 11 (KLHL11) who is a known member of the cullin-RING-based BCR (BTB-CUL3-RBX1) E3 ubiquitin-protein ligase complex. KLHL11 expression levels are capable of distinguishing LD from healthy controls well, but not as well in distinguishing acute LD from PTLD. Another gene, Undifferentiated Embryonic Cell Transcription Factor 1 (UTF1), can distinguish acute LD from PTLD well, but does not perform well in classifying LD from healthy controls. A classifier that combines KLHL11 and UTF1 together performs much better than a random classifier, but not well enough to become a reliable diagnostic (FIG. 7A). Additionally, the performance of all singular marker genes on the four classification tasks and computed ROC and AUC scores were assessed in the same way on the test set (FIG. 7B).

### REFERENCES

1. Kugeler K J, Schwartz A M, Delorey M J, Mead P S, Hinckley A F: Estimating the frequency of Lyme disease diagnoses, United States, 2010-2018. Emerging Infectious Diseases 2021, 27 (2): 616.
2. Branda J A, Steere A C: Laboratory diagnosis of Lyme borreliosis. Clinical microbiology reviews 2021, 34 (2): e00018-00019.
3. Porwancher R, Landsberg L: Optimizing use of multi-antibody assays for Lyme disease diagnosis: A bioinformatic approach. PLOS One 2021, 16 (9): e0253514.
4. Wormser G P, Dattwyler R J, Shapiro E D, Halperin J J, Steere A C, Klempner M S, Krause P J, Bakken J S, Strle F, Stanek G et al: The clinical assessment, treatment, and prevention of lyme disease, human granulocytic anaplasmosis, and babesiosis: clinical practice guidelines by the Infectious Diseases Society of America. Clin Infect Dis 2006, 43 (9): 1089-1134.
5. Aucott J N, Yang T, Yoon I, Powell D, Geller S A, Rebman A W: Risk of post-treatment Lyme disease in patients with ideally-treated early Lyme disease: A prospective cohort study. Int J Infect Dis 2022, 116:230-237.
6. Bobe J R, Jutras B L, Horn E J, Embers M E, Bailey A, Moritz R L, Zhang Y, Soloski M J, Ostfeld R S, Marconi R T et al: Recent Progress in Lyme Disease and Remaining Challenges. Front Med (Lausanne) 2021, 8:666554.
7. Bouquet J, Soloski M J, Swei A, Cheadle C, Federman S, Billaud J-N, Rebman A W, Kabre B, Halpert R, Boorgula M: Longitudinal transcriptome analysis reveals a sustained differential gene expression signature in patients treated for acute Lyme disease. MBio 2016, 7 (1): e00100-00116.
8. Clarke D J, Rebman A W, Bailey A, Wojciechowicz M L, Jenkins S L, Evangelista J E, Danieletto M, Fan J, Eshoo M W, Mosel M R: Predicting Lyme Disease From Patients' Peripheral Blood Mononuclear Cells Profiled With RNA-Sequencing. Frontiers in Immunology 2021, 12:452.
9. Petzke M M, Volyanskyy K, Mao Y, Arevalo B, Zohn R, Quituisaca J, Wormser G P, Dimitrova N, Schwartz I: Global transcriptome analysis identifies a diagnostic signature for early disseminated Lyme disease and its resolution. Mbio 2020, 11 (2): e00047-00020.
10. Marques A, Schwartz I, Wormser G P, Wang Y, Hornung R L, Demirkale C Y, Munson P J, Turk S-P, Williams C, Lee C-C R: Transcriptome assessment of erythema migrans skin lesions in patients with early Lyme disease reveals predominant interferon signaling. The Journal of infectious diseases 2018, 217 (1): 158-167.
11. Jiang R, Meng H, Raddassi K, Fleming I, Hoehn K B, Dardick K R, Belperron A A, Montgomery R R, Shalek A K, Hafler D A: Single-cell immunophenotyping of the skin lesion erythema migrans identifies IgM memory B cells. JCI insight 2021, 6 (12).
12. Kuleshov M V, Jones M R, Rouillard A D, Fernandez N F, Duan Q, Wang Z, Koplev S, Jenkins S L, Jagodnik K M, Lachmann A et al: Enrichr: a comprehensive gene set enrichment analysis web server 2016 update. Nucleic Acids Res 2016, 44 (W1): W90-97.
13. Piñero J, Queralt-Rosinach N, Bravo A, Deu-Pons J, Bauer-Mehren A, Baron M, Sanz F, Furlong L I: DisGeNET: a discovery platform for the dynamical exploration of human diseases and their genes. Database 2015, 2015.
14. Hamosh A, Scott A F, Amberger J S, Bocchini C A, McKusick V A: Online Mendelian Inheritance in Man (OMIM), a knowledgebase of human genes and genetic disorders. Nucleic acids research 2005, 33 (suppl_1): D514-D517.
15. Matera G, Labate A, Quirino A, Lamberti A G, BorzÃ G, Barreca G S, Mumoli L, Peronace C, Giancotti A, Gambardella A et al: Chronic neuroborreliosis by B. garinii: an unusual case presenting with epilepsy and multifocal brain MRI lesions. New Microbiol 2014, 37 (3): 393-397.
16. Juric S, Janculjak D, Tomic S, Butkovic Soldo S, Bilic E: Epileptic seizure as initial and only manifestation of neuroborreliosis: case report. Neurol Sci 2014, 35 (5): 793-794.
17. Morrissette M, Pitt N, González A, Strandwitz P, Caboni M, Rebman A W, Knight R, D′onofrio A, Aucott J N, Soloski M J: A distinct microbiome signature in posttreatment Lyme disease patients. MBio 2020, 11 (5): e02310-02320.
18. Fitzgerald B L, Graham B, Delorey M J, Pegalajar-Jurado A, Islam M N, Wormser G P, Aucott J N, Rebman A W, Soloski M J, Belisle J T: Metabolic response in patients with post-treatment Lyme disease symptoms/syndrome. Clinical Infectious Diseases 2021, 73 (7): e2342-e2349.
19. Aucott J N, Soloski M J, Rebman A W, Crowder L A, Lahey L J, Wagner C A, Robinson W H, Bechtold K T: CCL19 as a chemokine risk factor for posttreatment Lyme disease syndrome: a prospective clinical cohort study. Clinical and Vaccine Immunology 2016, 23 (9): 757-766.
20. Kanehisa M, Goto S: KEGG: kyoto encyclopedia of genes and genomes. Nucleic acids research 2000, 28 (1): 27-30.
21. Consortium G O: The gene ontology resource: 20 years and still Going strong. Nucleic acids research 2019, 47 (D1): D330-D338.
22. Slenter D N, Kutmon M, Hanspers K, Riutta A, Windsor J, Nunes N, Mélius J, Cirillo E, Coort S L, Digles D: WikiPathways: a multifaceted pathway database bridging metabolomics to other omics research. Nucleic acids research 2018, 46 (D1): D661-D667.
23. Huang R, Grishagin I, Wang Y, Zhao T, Greene J, Obenauer J C, Ngan D, Nguyen D-T, Guha R, Jadhav A: The NCATS BioPlanet—an integrated platform for exploring the universe of cellular signaling pathways for toxicology, systems biology, and chemical genomics. Frontiers in pharmacology 2019:445.
24. Liberzon A, Subramanian A, Pinchback R, Thorvaldsdóttir H, Tamayo P, Mesirov J P: Molecular signatures database (MSigDB) 3.0. Bioinformatics 2011, 27 (12): 1739-1740.
25. Rebman A W, Bechtold K T, Yang T, Mihm E A, Soloski M J, Novak C B, Aucott J N: The Clinical, Symptom, and Quality-of-Life Characterization of a Well-Defined Group of Patients with Posttreatment Lyme Disease Syndrome. Front Med (Lausanne) 2017, 4:224.
26. Dobin A, Davis C A, Schlesinger F, Drenkow J, Zaleski C, Jha S, Batut P, Chaisson M, Gingeras T R: STAR: ultrafast universal RNA-seq aligner. Bioinformatics 2013, 29 (1): 15-21.
27. Robinson M D, McCarthy D J, Smyth G K: edgeR: a Bioconductor package for differential expression analysis of digital gene expression data. Bioinformatics 2010, 26 (1): 139-140.
28. Law C W, Chen Y, Shi W, Smyth G K: voom: Precision weights unlock linear model analysis tools for RNA-seq read counts. Genome biology 2014, 15 (2): 1-17.
29. Smyth G K: Limma: linear models for microarray data. In: Bioinformatics and computational biology solutions using R and Bioconductor. Springer; 2005:397-420.
30. Consortium TGO: The Gene Ontology Resource: 20 years and still Going strong. Nucleic Acids Res 2019, 47 (D1): D330-d338.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of detecting Lyme disease or post-treatment Lyme disease in a subject in need thereof, the method comprising:
identifying changes in expression levels of at least one mRNA present in a biological sample, wherein the biological sample was obtained from a subject having, suspected of having, or at risk of having Lyme disease or post-treatment Lyme disease;
comparing the at least one mRNA's expression level with the levels of the same mRNA of a reference sample or control sample; and
wherein changes in the expression levels of the subject's mRNA from those of the reference sample or control sample correlates with a diagnosis of Lyme disease or post-treatment Lyme disease.

2. The method of claim 1, further comprising calculating a score for the at least one mRNA and assigning a classification to the at least one mRNA based on the score.

3. The method of claim 2, further comprising predicting a likelihood of the subject having Lyme disease or post-treatment Lyme disease based on the score.

4. A computer-implemented method for detecting Lyme disease or post-treatment Lyme disease in a subject in need thereof, the method comprising:
obtaining mRNA expression data for a biological sample, wherein the biological sample was obtained from a subject having, suspected of having, or at risk of having Lyme disease or post-treatment Lyme disease;
identifying, with one or more computing devices, changes in expression levels of at least one mRNA present in a biological sample;
comparing, with one or more computing devices, the at least one mRNA's expression level with the levels of the same mRNA of a reference sample or control sample; and
calculating, with one or more computing devices, a likelihood of the subject having Lyme disease or post-treatment Lyme disease based on the comparison of the expression levels.

5. The method of claim 4, further comprising using one or more computing devices to assign a threshold value to the at least one mRNA.

6. The method of claim 4, wherein the identifying, comparing, and/or calculating step may be modified based on at least one machine learning algorithm.

7. The method of claim 1, wherein the at least one mRNA is an upregulated mRNA or a downregulated mRNA.

8. The method of claim 1, wherein the biological sample is selected from the group consisting of whole blood, peripheral blood mononuclear cells, plasma, serum, lymph, cerebrospinal fluid, ascites, and tissue biopsy.

9. The method of claim 1, wherein the control sample is from a healthy subject or a subject with acute Lyme disease.

10. The method of claim 1, wherein the mRNA is selected from the group consisting of KLHL11, UTF1, NBPF1, RBMS3, PPFIA4, NOTCH3, SLC4A10, TMEM272, CBARP, MLANA, CHDH, NAPIL2, TMEM52B, C2CD4D, OTUB2, POMK, KCNG1, CAND2, DCSTAMP, NBEAL1, SCN3A, AHNAK2, RAD50, APBA1, DNAH7, CXADR, AMH, ALDH7A1, CACNB4, CMTIV11, SHANK1, TULP2, NEK5, BTNL9, and GPR135.

11. The method of claim 1, wherein the method comprises identifying changes in expression levels of at least two mRNAs.

12. The method of claim 10, wherein the method comprises identifying changes in expression levels of KLHL11 and UTF1.

13. A system for detecting Lyme disease or post-treatment Lyme disease in a subject in need thereof, the system comprising:
one or more computing devices, and
a memory having instructions stored thereon, wherein the instructions, when executed by the one or more computing devices, cause the one or more computing devices to:
identify changes in expression levels of at least one mRNA present in a biological sample, wherein the biological sample was obtained from a subject having, suspected of having, or at risk of having Lyme disease or post-treatment Lyme disease;
compare the at least one mRNA's expression level with the levels of the same mRNA of a reference sample or control sample; and
calculate a likelihood of the subject having Lyme disease or post-treatment Lyme disease based on the comparison of the expression levels.

14. The system of claim 13, wherein the system is configured to provide defined artificial intelligence (AI) sensing and autonomous response.

15. The system of claim 13, wherein the at least one mRNA is an upregulated mRNA or a downregulated mRNA.

16. The system claim 13, wherein the biological sample is selected from the group consisting of whole blood, plasma, peripheral blood mononuclear cells, serum, lymph, cerebrospinal fluid, ascites, and tissue biopsy.

17. The system claim 13, wherein the control sample is from a healthy subject or a subject with acute Lyme disease.

18. The system of claim 13, wherein the mRNA is selected from the group consisting of KLHL11, UTF1, NBPF1, RBMS3, PPFIA4, NOTCH3, SLC4A10, TMEM272, CBARP, MLANA, CHDH, NAP1L2, TMEM52B, C2CD4D, OTUB2, POMK, KCNG1, CAND2, DCSTAMP, NBEAL1, SCN3A, AHNAK2, RAD50, APBA1, DNAH7, CXADR, AMH, ALDH7A1, CACNB4, CMTM1, SHANK1, TULP2, NEK5, BTNL9, and GPR135.

19. The system of claim 13, wherein the system comprises identifying changes in expression levels of at least two mRNAs.

20. The system of claim 18, wherein the system comprises identifying changes in expression levels of KLHL11 and UTF1.

21. (canceled)
